(19) United States
(12) Patent Application Publication
(10) Pub. No.: US 2023/0310647 A1
(43) Pub. Date: Oct. 5, 2023

(54) TARGETING CARTILAGE EGFR PATHWAY FOR OSTEOARTHRITIS TREATMENT

(71) Applicant: THE TRUSTEES OF THE UNIVERSITY OF PENNSYLVANIA, Philadelphia, PA (US)

(72) Inventors: Zhiliang CHENG, Newtown, PA (US); Ling QIN, Moorestown, NJ (US); Yulong WEI, Hubei (CN)

(21) Appl. No.: 18/042,092

(22) PCT Filed: Aug. 12, 2021
(86) PCT No.: PCT/US2021/045721

§ 371 Date: Feb. 17, 2023

Related U.S. Application Data
(60) Provisional application No. 63/067,546, filed on Aug. 19, 2020.

(51) Int. Cl. A61K 47/69; A61P 19/02

(52) U.S. Cl. CPC A61K 47/6937; A61P 19/02

(57) ABSTRACT

Provided are therapeutic compositions, comprising: a polymeric nanoparticle; a ligand selected to activate an EGFR receptor; and a linker, the linker associating the nanoparticle and the ligand. Also provided are therapeutic compositions, comprising: a nanoparticle; a ligand, the ligand being any one of EGF, transforming growth factor-alpha (TGFα), heparin-binding EGF-like growth factor (HBEGF), betacellulin (BTC), amphiregulin (AREG), epiregulin (EREG), or epigen; and a linker associating the nanoparticle and the ligand, the therapeutic composition having a surface charge in the range of from about −5 to about 30 mV. Related methods of treatment are also provided.

### RELATED APPLICATIONS

The present application claims priority to and the benefit of U.S. patent application Ser. No. 63/067,546, “Targeting Cartilage EGFR Pathway For Osteoarthritis Treatment” (filed Aug. 19, 2020), the entirety of which application is incorporated herein by reference for any and all purposes.

### GOVERNMENT RIGHTS

This invention was made with government support under AR066098, AR074570, and DK095803 awarded by the National Institutes of Health. The government has certain rights in the invention.

### TECHNICAL FIELD

The present disclosure relates to the field of cartilage growth modulation and to the field of nanoparticulate delivery systems.

### BACKGROUND

Osteoarthritis (OA) is the most common chronic condition of the joints, affecting approximately 15% of people worldwide (i.e., about 630 million). As a joint degenerative disease, it is primarily characterized by destruction of articular cartilage, but is often accompanied by subchondral bone thickening, osteophyte formation, synovial inflammation, and hypertrophy of the joint capsule (1). An accelerated form of OA after articular injury, post traumatic osteoarthritis, affects additional individuals with a more acute form of degeneration.

Despite the fact that OA patients have a great level of pain and disability, neither a cure nor a disease-modifying treatment exists. Accordingly, there is a long-felt need in the art for an improved OA treatment.

### SUMMARY

In meeting the described long-felt needs, the present disclosure provides therapeutic compositions, comprising: a polymeric nanoparticle; a ligand selected to activate an EGFR receptor; and a linker, the linker associating the nanoparticle and the ligand.

Also provided are therapeutic compositions, comprising: a nanoparticle; a ligand, the ligand being any one of EGF, transforming growth factor-alpha (TGFα), heparin-binding EGF-like growth factor (HBEGF), betacellulin (BTC), amphiregulin (AREG), epiregulin (EREG), or epigen; and a linker associating the nanoparticle and the ligand, the therapeutic composition having a surface charge in the range of from about −5 to about 30 mV.

Further disclosed are methods of treating joint pain in a patient in need thereof, the method comprising: administering to the patient a therapeutically effective amount of a composition comprising a therapeutic composition as disclosed herein.

Also provided are pharmaceutically acceptable compositions, comprising a therapeutic composition as disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1A-1G illustrates that overexpression of HBEGF in chondrocytes expanded mouse growth plate and articular cartilage without affecting the gross appearance of knee joints.

FIGS. 2A-2K illustrate that overexpression of HBEGF increased chondroprogenitors in articular cartilage.

FIGS. 3A-3G illustrates that overexpressing HBEGF in articular cartilage delayed OA progression.

FIGS. 4A-4D illustrate that the protective action of HBEGF overexpression on articular cartilage during OA development was EGFR-dependent.

FIGS. 5A-5I illustrate exemplary preparation and characterization of TGFα-NPs.

FIGS. 6A-6F illustrate that TGFα-NPs exhibited full length penetration of human-thickness bovine articular cartilage and extend residence time in both healthy and diseased knee joints.

FIGS. 7A-7I illustrate that TGFα-NP treatment attenuated OA progression after DMM surgery.

FIGS. 8A-8B illustrate that HBEGF Over^{Col2} mice had normal body weight and body length.

FIGS. 9A-9B. illustrate that overexpressing HBEGF in articular cartilage did not affect bone structure.

FIGS. 10A-1 B illustrate that overexpressing HBEGF in cartilage did not affect cartilage matrix composition and cartilage degradation.

FIGS. 11A-11B illustrate that overexpressing HBEGF in cartilage did not affect vital internal organs.

FIGS. 12A-12B illustrate that HBEGF Over^{AgcER} mice had increased HBEGF expression and EGFR activity in knee articular cartilage.

FIGS. 13A-13B provide a chemical structure (FIG. 13A) and ¹H NMR spectrum (FIG. 13B) of PLL-PCL.

FIG. 14 illustrates that TGFα-NPs resulted in similar morphology changes in chondrocytes as free TGFα.

FIGS. 15A-15B illustrate that TGFα-NPs doped with PLL-PCL enhanced bovine cartilage uptake.

FIGS. 16A-16F illustrate that TGFα-NPs doped with PLL-PCL improved their penetration and retention in the bovine cartilage tissue.

FIGS. 17A-17D illustrate example biodistribution of TGFα-NPs within the knee joints and some major organs.

FIGS. 18A-18B illustrate the OA severity of knee joints (FIG. 18A) and uncalcified cartilage thickness (FIG. 18B) are measured in mice with PBS, TGFα-DBCO, Ctrl-NP and TGFα-NP treatment at 2 months post-surgery.

FIGS. 19A-19C illustrate that intra-articular injections of TGFα-NPs into cartilage did not affect vital internal organs and gross joint morphology.

Table 1. Mouse real-time PCR primer sequences.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints, 2 grams and 10 grams, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

Osteoarthritis (OA) is a widespread joint disease currently with no disease-modifying treatments. Our previous studies revealed that mice with cartilage-specific EGFR deficiency develop accelerated knee OA under normal and injury conditions. To test whether cartilage EGFR pathway can be targeted as a novel OA therapy, we constructed two cartilage-specific EGFR over-activation models by overexpressing HBEGF, an EGFR ligand. Compared to WT, Col2-Cre HBEGFOver mice had persistently enlarged articular cartilage from adolescence, due to an expanded pool of chondroprogenitors with elevated proliferation ability, survival rate, and lubricant production. Strikingly, adult Col2-Cre HBEGFOver mice and Aggrecan-CreER HBEGFOver mice were resistant to cartilage degeneration and other signs of OA symptoms after OA surgery. Treating mice with Gefitinib, an EGFR inhibitor, abolished the protective action against OA in HBEGFOver mice. To pharmacologically target EGFR, we conjugated TGFα, a potent EGFR ligand, to polymeric micellar nanoparticles (NPs). The resultant TGFα-NPs were stable, non-toxic, possessed long joint retention, high cartilage uptake and penetration capabilities. Intra-articular delivery of TGFα-NPs effectively attenuated surgery-induced OA cartilage degeneration, subchondral bone plate sclerosis, and joint pain. Genetic or pharmacologic activation of EGFR revealed no obvious side effects in knee joints and major vital organs in mice. Taken together, our studies demonstrate the feasibility of targeting EGFR signaling for OA treatment as a novel therapeutic approach using nanotechnology.

Overexpressing HBEGF in chondrocytes leads to cartilage enlargement

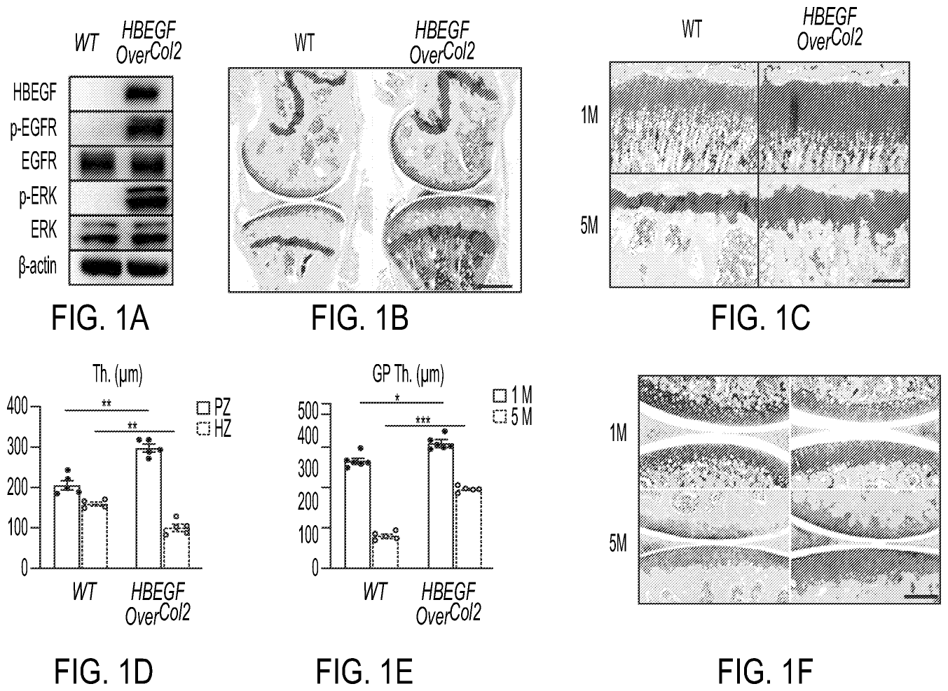

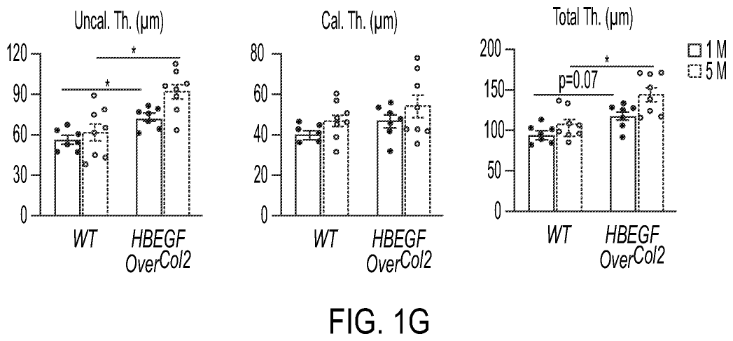

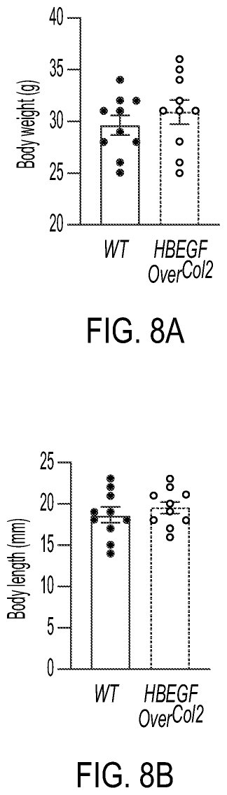

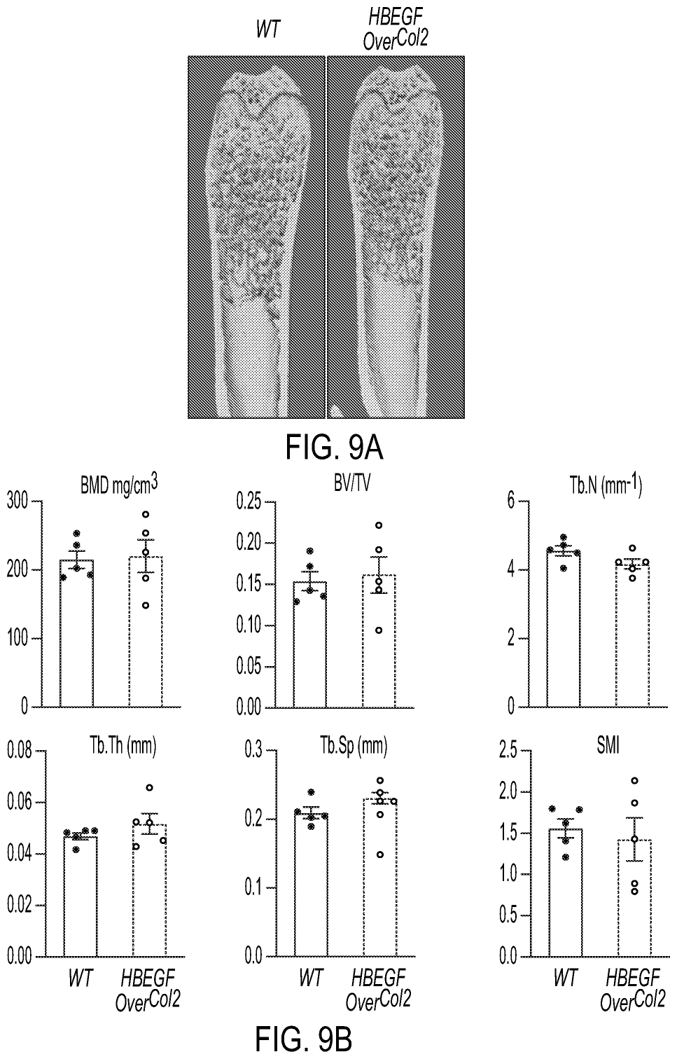

To target EGFR pathway in the cartilage, we generated Col2-Cre Rosa DTR mice, i.e. Col1-Cre Rosa-HBEGF (HBEGF Over^{Col2}) mice. These mice had similar body weight and body length as WT (FIG. 8). Western blots confirmed increased HBEGF amount in cartilage chondrocytes, leading to EGFR activation as shown by elevated p-EGFR and p-ERK levels (FIG. 1A). At 5 months of age, HBEGF Over^{Col2} mice displayed normal knee joints without any gross abnormality, such as osteophyte and synovitis (FIG. 1B). Long bone structure, particularly metaphyseal trabecular bone, was also not affected (FIGS. 9A-9B).

The most obvious change in the HBEGF Over^{Col2} skeleton is cartilage. At 1 month of age, its growth plate was modestly expanded by 16%, mainly due to the elongation of the proliferative zone (FIG. 1C-1E). On the contrary, its hypertrophic zone was shrunk. At 5 months of age, the expansion of growth plate was more obvious (2.41-fold). Similar expansion was also observed in articular cartilage (23% and 34% increases in total cartilage thickness at 1 and 5 months of age, respectively, FIG. 1F and FIG. 1G). Detailed analysis revealed a significant thickness increase in uncalcified cartilage but not in calcified cartilage.

EGFR over-activation expands the chondroprogenitor pool

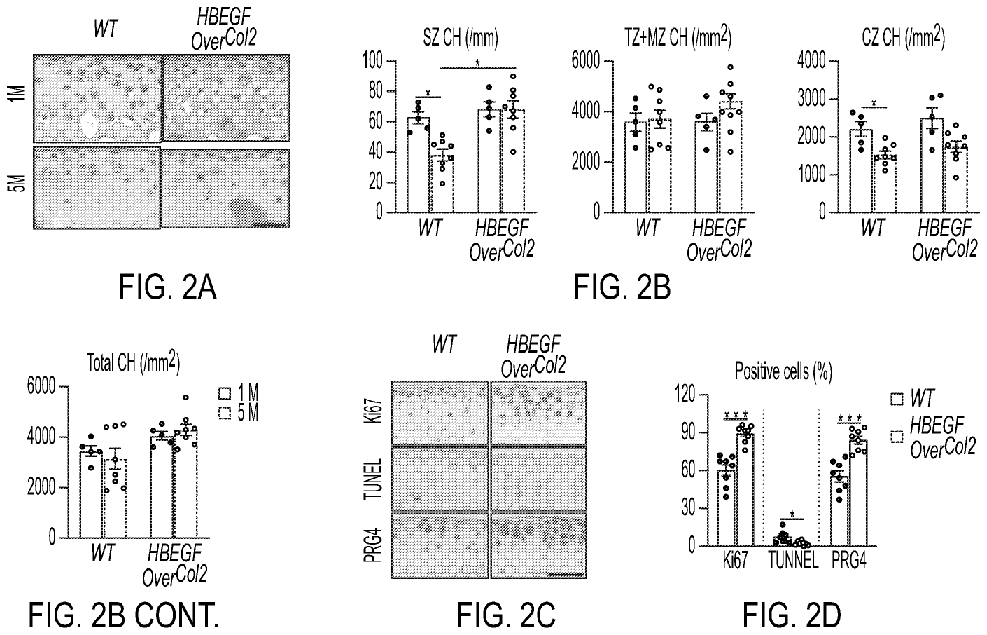

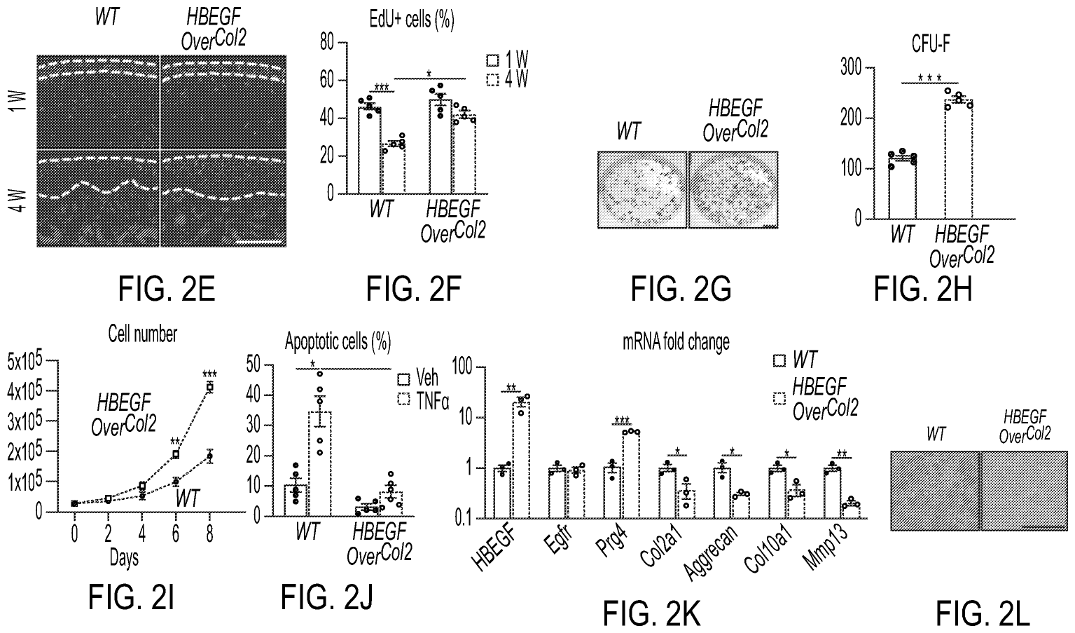

The superficial layer contains chondroprogenitors responsible for forming cells in the rest of articular cartilage during development. A previous study showed that EGFR inactivation in chondrocytes (Col2-Cre CKO) leads to fewer superficial chondrocytes. In WT joints, the number of chondrocytes in the superficial zone declined by 39% during cartilage maturation (FIG. 2A-2B). Interestingly, this decline did not occur in HBEGF Over^{Col2} mice, which exhibited a 1.79-fold increase in superficial chondrocytes compared to WT at 5 months of age (FIG. 2A-2B). Cellularity in transitional/middle zone and calcified zone of HBEGF Over^{Col2} mice also showed an upward trend compared to WT (FIG. 2A-2B). This was accompanied by enhanced Ki67 and Prg4 staining and reduced TUNEL staining (FIG. 2C-2D), suggesting that constitutive over-activation of EGFR signaling promotes proliferation, survival and lubricant synthesis in chondrocytes.

EdU labels cells undergoing proliferation

At 1 week of age, 46% of periarticular cells in epiphyseal cartilage, the site for future articular cartilage, was labeled in WT mice (FIG. 2E-2F). Three weeks later when articular cartilage is established, 27% of chondrocytes (most of them in uncalcified cartilage) were still labeled by EdU, indicating that many proliferative chondrocytes are slow-cycling cells, i.e. chondroprogenitors. HBEGF Over^{Col2} mice possessed more EdU⁺ cells than WT mice at 4 weeks of age (FIG. 2E-2F). After dissection and digestion, 5-month-old HBEGF Over^{Col2} cartilage formed 1.96-fold more CFU-F colonies than WT cartilage in culture (FIG. 2G-2H). In addition, progenitors from HBEGF Over^{Col2} cartilage grew much quicker than those from WT (FIG. 2I) and were resistant to TNFα-induced apoptosis (FIG. 2J). Taken together, our in vivo and in vitro data demonstrated that HBEGF overexpression produces more chondroprogenitors in articular cartilage with superior proliferation and survival abilities.

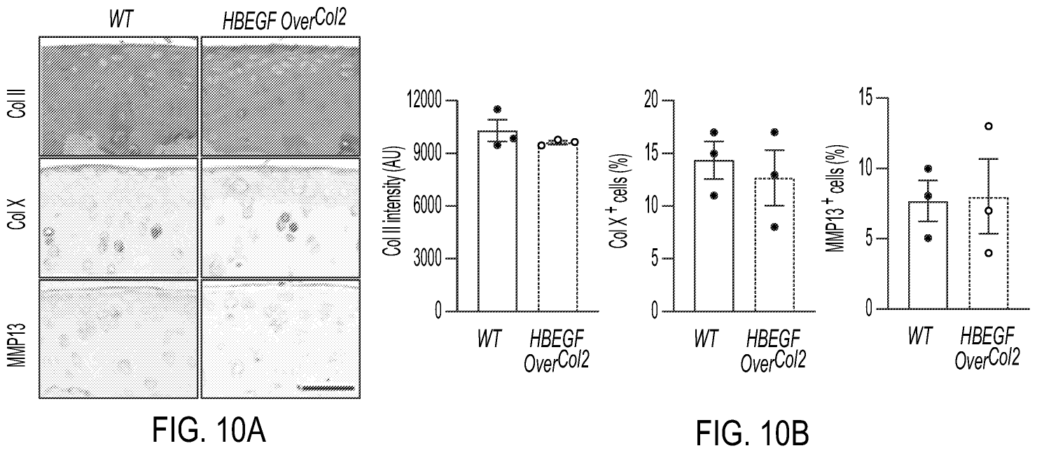

When subjected to chondrogenic differentiation, progenitors from HBEGF Over^{Col2} cartilage expressed more Prg4 but less cartilage matrix (Aggrecan, Col1aI, and ColI0aI) and proteases (Mmp13, FIG. 2K). They were able to differentiate into Alcian Blue positive cartilage albeit the staining intensity was less than WT (FIG. 2L). While these in vitro data indicate that overexpression of HBEGF modestly decreases chondrogenic differentiation, immunostaining clearly showed that proteoglycan (FIG. 1F), type II collagen, type X collagen, and MMP13 (FIGS. 10A-10B) amounts are not altered in HBEGF Over^{Col2} cartilage, suggesting that over-activation of EGFR signaling does not negatively affect cartilage components in vivo.

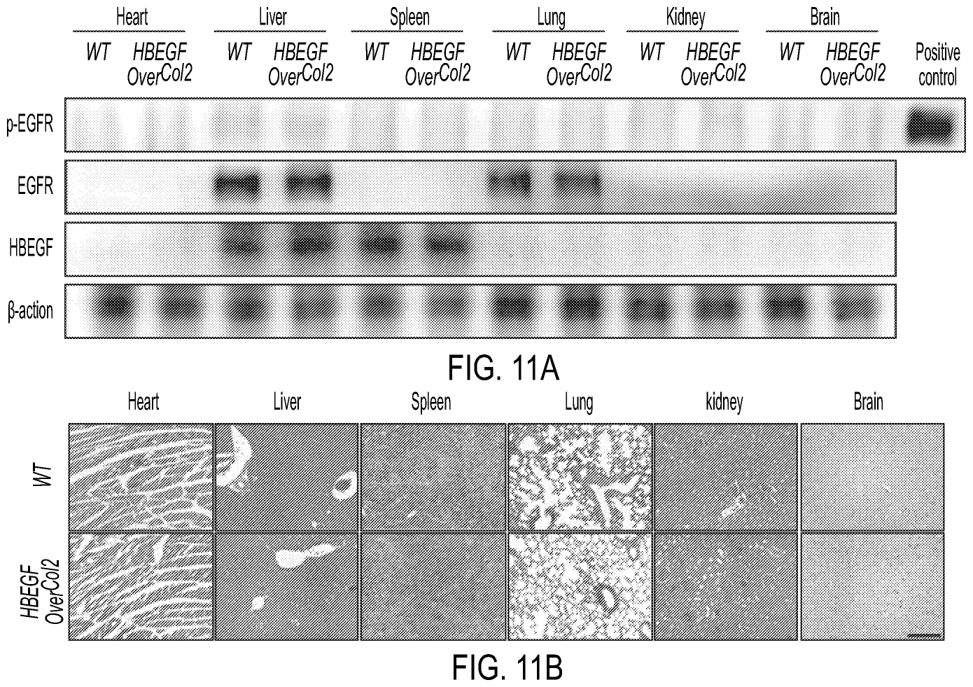

As a transmembrane protein, HBEGF is cleaved by a sheddase and released from the cell membrane for paracrine and systemic actions. Because EGFR is important for the development and homeostasis of multiple organs, a concern is raised about possible side effects of constitutively expressing HBEGF. However, we did not observe a detectable level of p-EGFR in major organs, such as heart, liver, spleen, lung, kidney, and brain from adult HBEGF Over^{Col2} mice (FIG. 11A). The endogenous levels of EGFR and HBEGF were also not altered (FIG. 11A). Most importantly, the morphology of these vital organs remained the same as WT mice (FIG. 11B), indicating no substantial side effects of cartilage-specific HBEGF overexpression.

EGFR over-activation attenuates OA progression

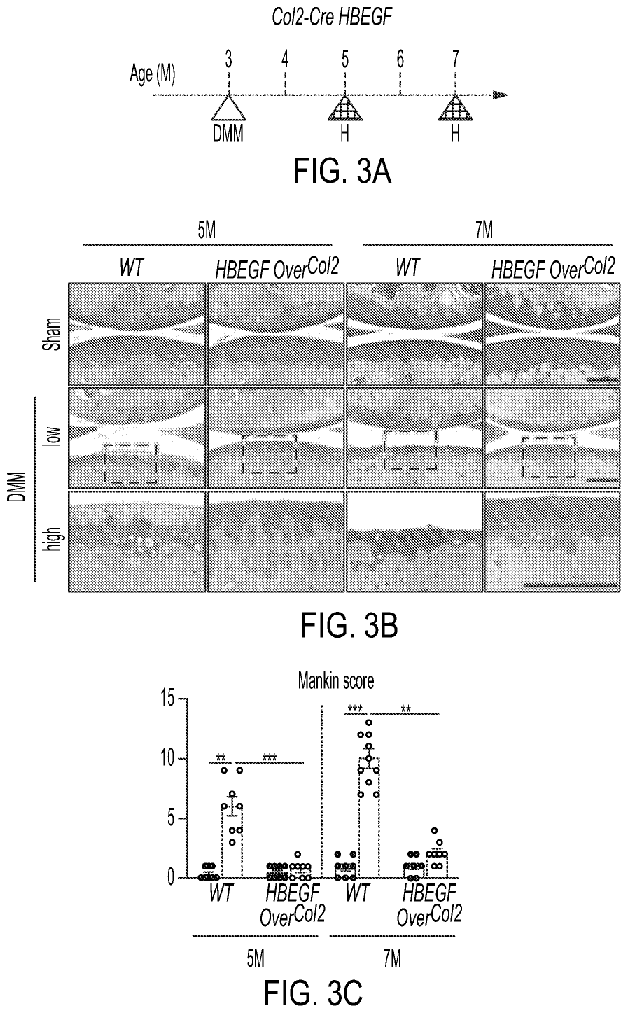

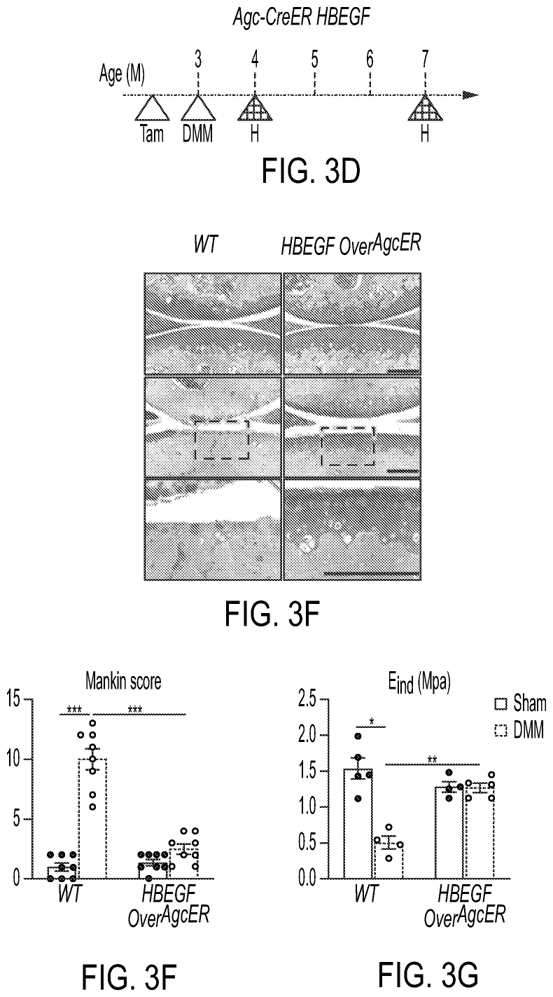

Next studied was the effect of HBEGF overexpression on OA progression induced by surgical destabilization of the medial meniscus (DMM) (FIG. 3A). At 2 months post-surgery, WT knees started to lose proteoglycan and exhibit fibrillation at the cartilage surface (FIG. 3B-3C). At 4 months post-surgery, they displayed severe cartilage erosion beyond the tidemark, accompanied by uneven cartilage surface or clefts. On contrast, in HBEGF Over^{Col2} mice, DMM knees showed only a minor loss in proteoglycan content at 2 months post-surgery. Two months later, the articular surface was still intact albeit their cartilage was thinner than that in sham knees. Quantifying OA severity at 4 months post-surgery revealed that overexpression of HBEGF reduces Mankin score from 10.0 to 2.1 at this stage (FIG. 3C). These data provided the first in vivo evidence that over-activation of EGFR could protect cartilage from degeneration upon OA inducing insults.

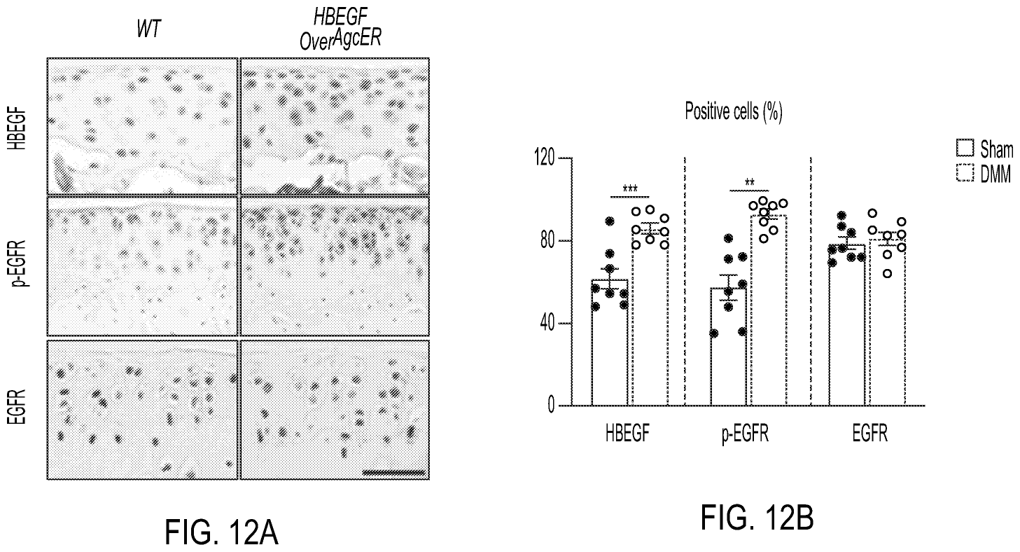

To eliminate the developmental effect in HBEGF Over^{Col2} mice, we next constructed an inducible model Aggrecan-CreER DTR (HBEGF Over^{AgcER}). Since Tamoxifen was injected right before DMM surgery (FIG. 3D), these mice had normal articular cartilage before injury. IHC confirmed that they have higher amounts of HBEGF and p-EGFR in articular cartilage compared to the sham knee at 1 month after induction (FIGS. 12A-12B). Four months after DMM surgery, while WT mice developed late OA with most cartilage eroded, HBEGF Over^{AgcER} maintained relatively intact articular cartilage with a low Mankin Score of 2.5 (FIG. 3E-3F). Our past study demonstrated that nano-indentation of cartilage surface is a sensitive method to detect early OA in mice. In line with this, the surface indentation modulus Erna was drastically reduced by 67% in WT cartilage at 1 month after DMM but remained unchanged in HBEGF Over^{AgcER} mouse knees (FIG. 3G), suggesting that overexpressing HBEGF in adult cartilage preserves the mechanical functions of cartilage surface after OA injury.

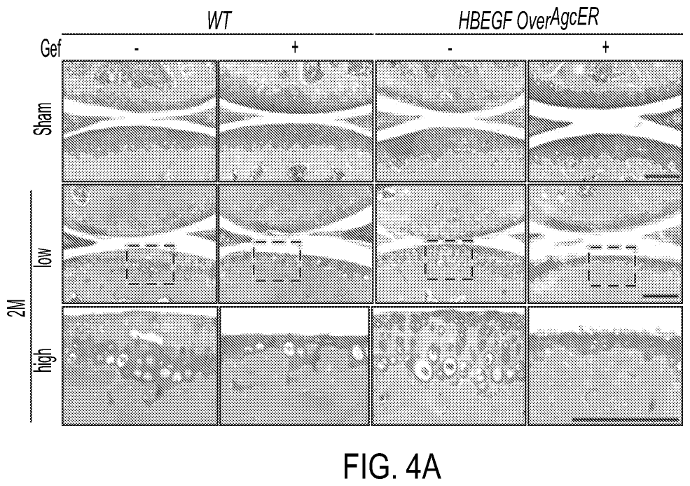

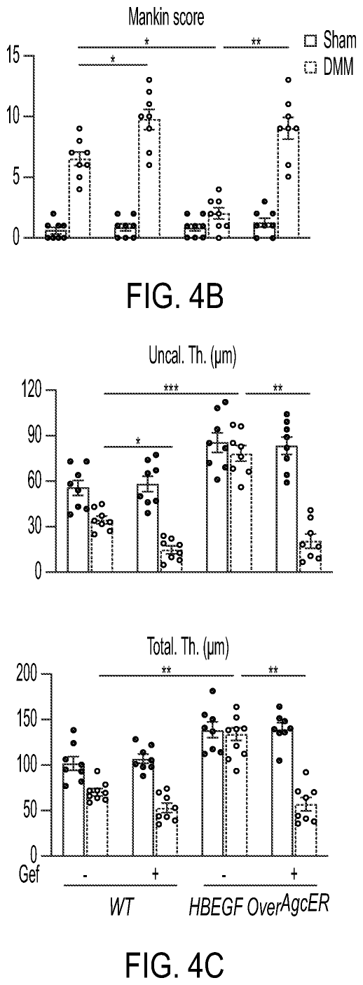

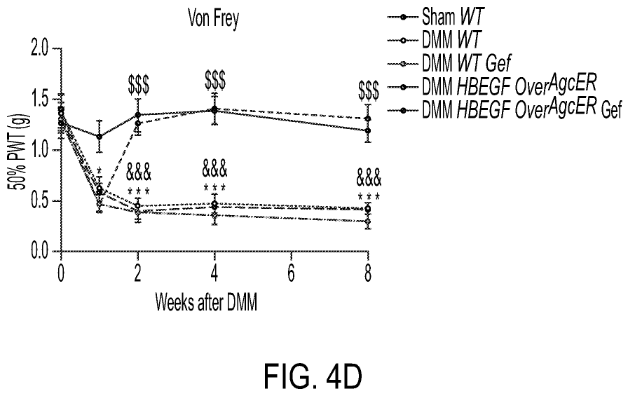

HBEGF binds and signals through EGFR as well as another EGFR family member, ErbB4. To study whether EGFR mediates the action of HBEGF on cartilage in vivo, we treated HBEGF Over^{AgcER} mice and WT controls with the EGFR-specific inhibitor Gefitinib once every other day after Tamoxifen induction and DMM surgery for 2 months. Similar to previous data, Gefitinib moderately accelerated OA progression in WT DMM knees, increasing Mankin score from 6.5 to 9.8 (FIG. 4A-4B). Strikingly, Gefitinib completely abolished the protective effect of HBEGF Over^{AgcER} on articular cartilage after DMM surgery, leading to marked cartilage erosion with elevated Mankin Score of 9.0. DMM mainly reduced the thickness of uncalcified cartilage, resulting 38% and 74% decreases in vehicle- and Gefitinib-treated WT mice, respectively (FIG. 4C). In HBEGF Over^{AgcER} mice, while DMM alone did not alter the cartilage thickness, co-treatment with Gefitinib greatly lessened the thicknesses of uncalcified and total cartilage by 75% and 59%, respectively (FIG. 4C). Furthermore, the von Frey behavioral pain test indicated that HBEGF Over^{AgcER} mice develop a similar level of pain as WT mice at 1 week after DMM but quickly recover to normal as sham mice, suggesting that overexpressing HBEGF also has functional benefits (FIG. 4D). However, Gefitinib abolished this effect. We did not detect any effect of this inhibitor on sham knees from WT or HBEGF Over^{AgcER} mice (data not shown). Therefore, our results indicated that HBEGF signals through EGFR in vivo to execute its chondroprotection action.

Synthesize and characterize TGFα-NPs

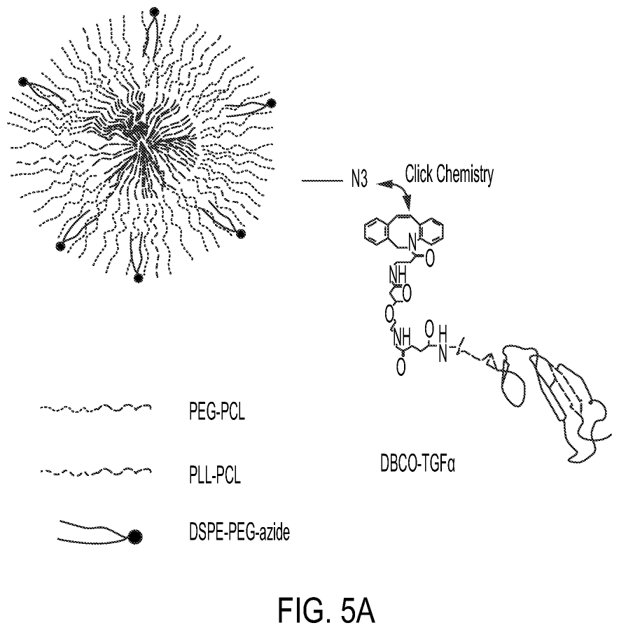

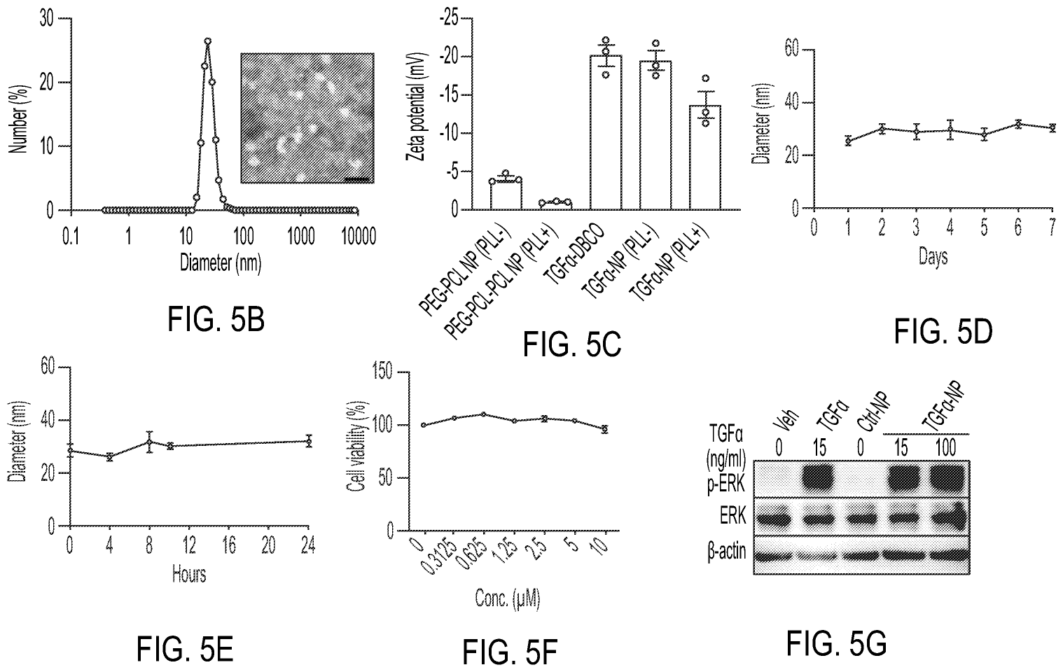

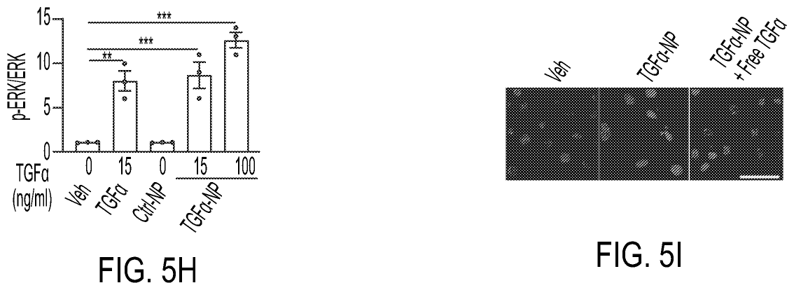

While cartilage does express TGFα and other EGFR ligands, the above mouse studies indicate endogenous EGFR ligand expression is not sufficient to protect cartilage against OA. To activate EGFR for OA treatment, we chose one of the most potent EGFR ligands, TGFα, as an activator. However, TGFα is not stable in the circulation and direct injection of it into knee joints suffers from rapid clearance due to its low molecular weight (5.6 kD). To overcome this challenge, we engineered a nanoparticle delivery system to prolong the retention of active TGFα in the knee joint. Briefly, bacteria-expressed human TGFα were synthesized and site-specifically labeled at the C-terminus with a constrained alkyne, dibenzocyclooctyne (DBCO), via sortase-tag expressed protein ligation (STEPL) (26, 27). TGFα-NPs were then prepared via copper-free click chemistry, by simply mixing TGFα-DBCO with azide-functionalized nanoparticles (FIG. 5A). Azide-functionalized nanoparticles were made from 55 mol % poly(ethylene glycol)-polycaprolactone (PEG-PCL)/20 mol % poly(L-lysine-block-poly(c-caprolactone) (PLL-PCL)/25 mol % 1,2-distearoyl-sn-glycero-3-phosphoethanolamine-N-[azido(polyethylene glycol)-5000](DSPE-PEG5K-N3) using the film hydration method.

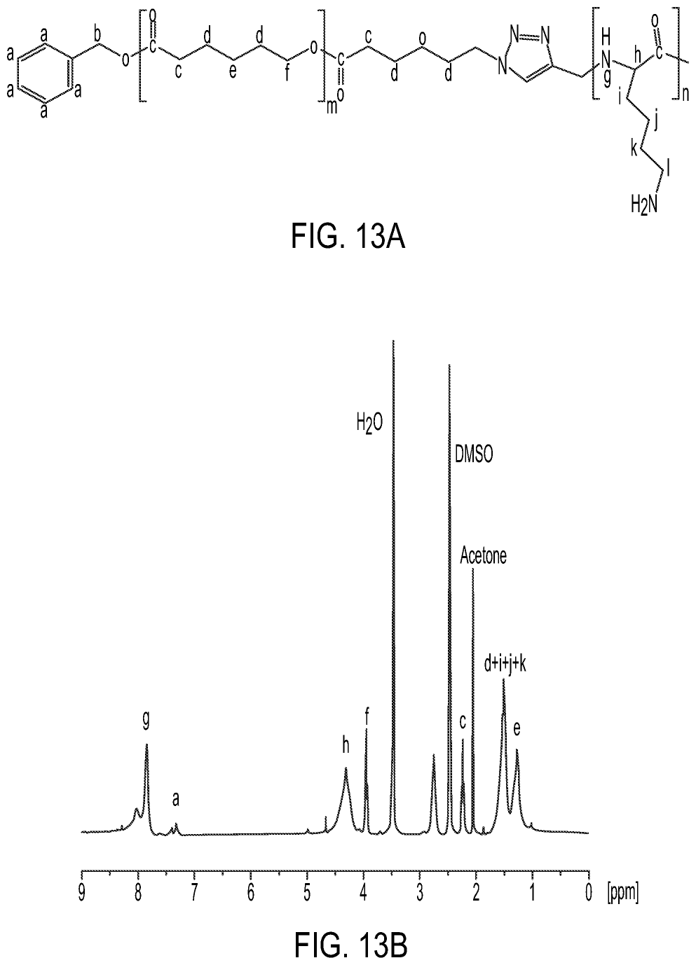

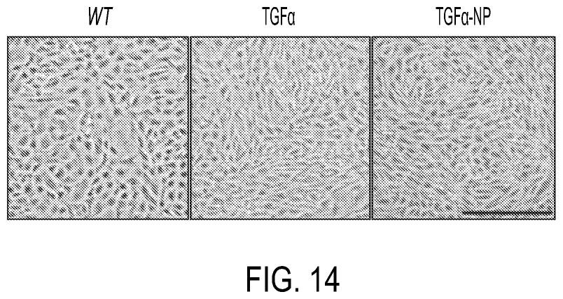

TGFα-NPs were approximately spherical in shape with a hydrodynamic diameter of 25.93 nm (FIG. 5B). Since nanoparticle surface charge could be adjusted to augment the interaction between the therapeutic agents and the anionic glycosaminoglycans (GAGs) in the cartilage, the cationic diblock copolymer PLL-PCL was synthesized (FIGS. 13A-13B) and introduced into the PEG-PCL nanoparticles to reduce their surface charge from −4.2 my to −1 my (FIG. 5C). Following the conjugation of TGFαthe surface charge of TGFα-NPs became more negative, mostly due to the negatively charged TGFα. However, the magnitude of the surface charge of TGFα-NPs was still reduced in nanoparticles containing PLL-PCL. For example, the surface charge of TGFα-NPs in the presence and absence PLL-PCL was −13.7 my and −19.4 my, respectively. We next characterized stability, cytotoxicity, and specificity of newly synthesized TGFα-NPs. We did not detect any change in the hydrodynamic diameter of TGFα-NPs in water for at least 1 week (FIG. 5D) or in bovine synovial fluid for 24 hours (FIG. 5E). MTT (3-(4,5-dimethylthiazol-2-yl)-2,5-diphenyltetrazolium bromide) assay showed that TGFα-NP treatment (up to 10 μM TGFα content) for 24 hours does not affect the cell viability of mouse primary chondrocytes (FIG. 5F). Western blot demonstrated that TGFα-NPs activate EGFR downstream target ERK as potent as free TGFα while Ctrl-NPs (nanoparticles with no TGFα conjugation) had no such effect (FIGS. 5G-5H). In addition, similar to free TGFα, TGFα-NPs changed chondrocytes from a polygonal cell shape to a more spindle cell shape after 2 days of treatment (FIG. 14). Using fluorescent rhodamine-labeled TGFα-NPs, we found that TGFα-NPs bound to the surface of primary chondrocytes in a TGFα-specific manner (FIG. 5I). Therefore, our data demonstrate that TGFα-NPs are stable, non-toxic, and functional.

TGFα-NPs have superior cartilage uptake, penetration, and joint retention abilities

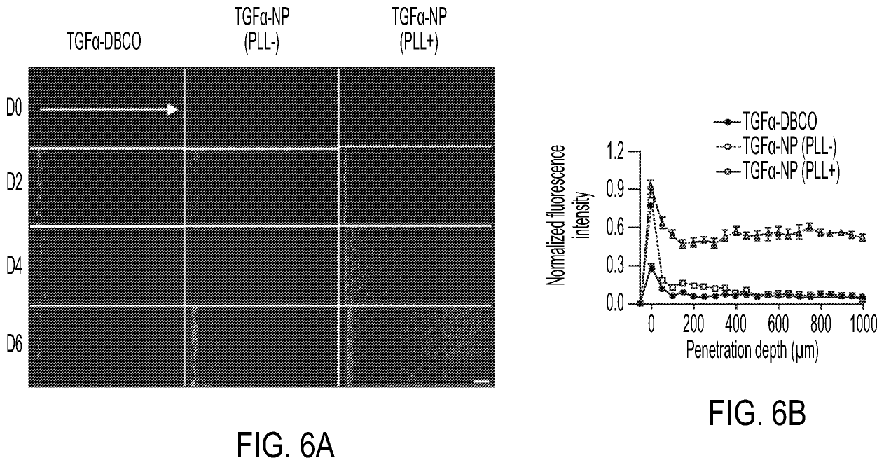

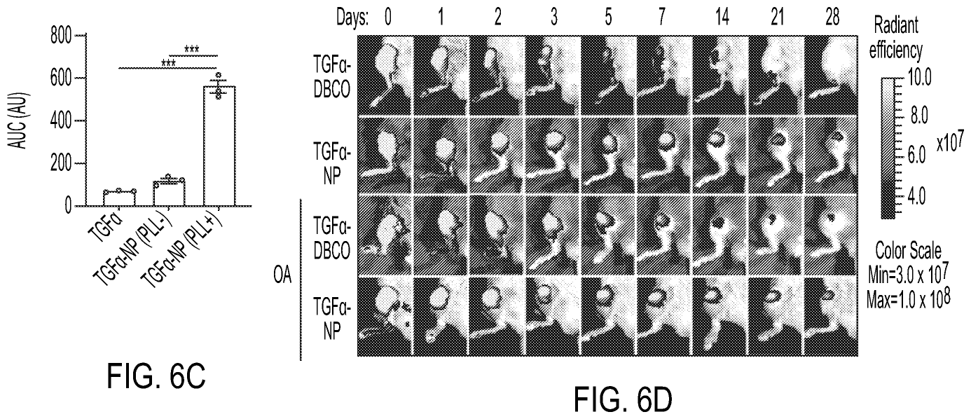

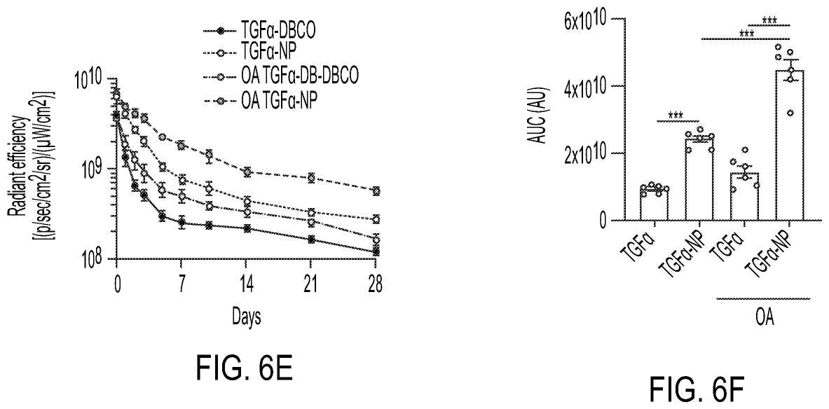

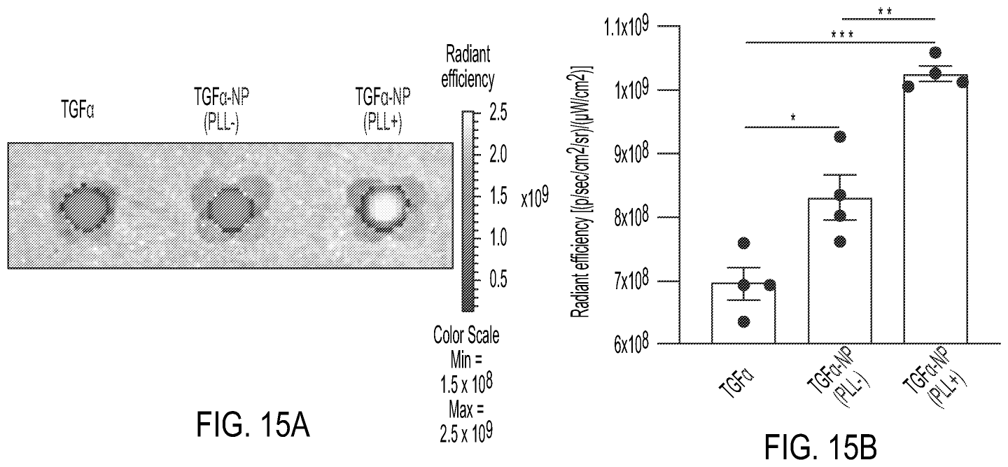

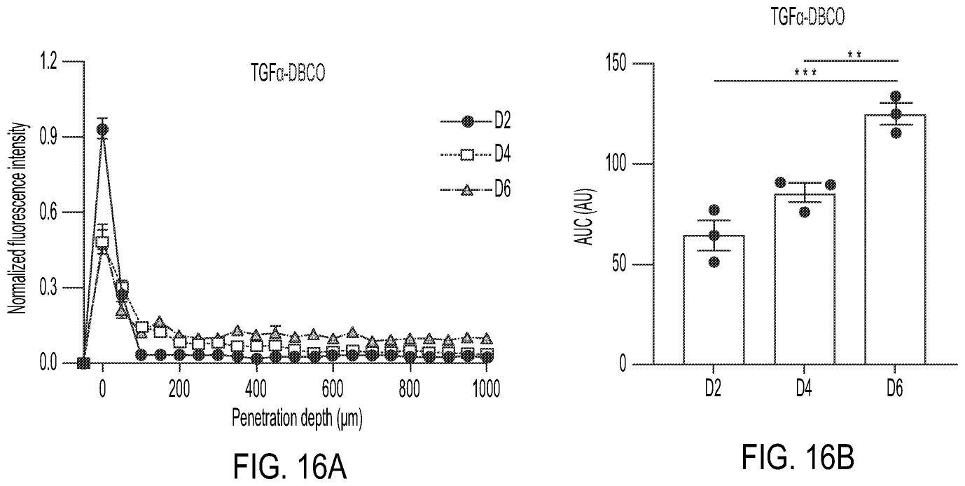

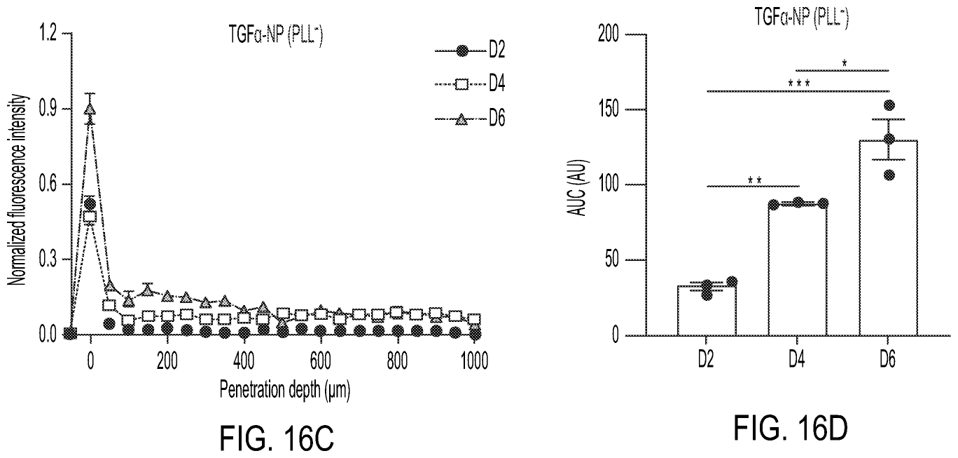

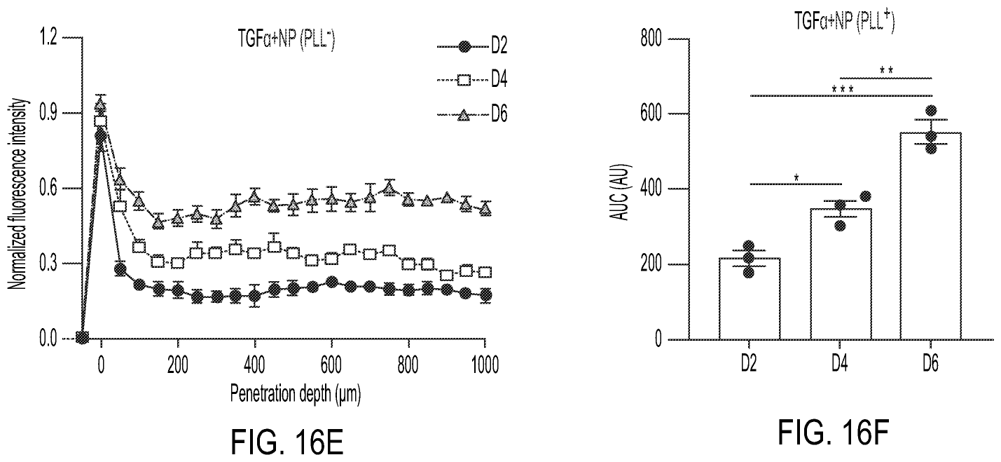

Human knee articular cartilage is about 2-4 mm thick and the superficial layer makes up 10-20% of cartilage thickness. To increase cartilage retention and to penetrate deeper into the cartilage, we doped cationic PLL-PCL into the nanoparticles to reduce their surface charge. Using a near infrared (NIR) fluorescence probe IRDye 800CW as a label, we found that bovine cartilage explants uptake much more PLL-PCL-doped TGFα-NPs than non-PLL-PCL-doped TGFα-NPs or TGFα-DBCO after a 24 hours incubation (FIGS. 15A-15B). To study penetration, we labeled TGFα-NPs and TGFα-DBCO with rhodamine. Strikingly, PLL-PCL-doped TGFα-NPs efficiently bound to the surface of bovine cartilage explant at day 2 and gradually penetrated inside at least 1 mm by day 6 (FIG. 6A-6B and FIG. 16E-16F). On the contrary, TGFα-DBCO and non-PLL-PCL-doped TGFα-NPs only accumulated at the cartilage surface but did not penetrate deep inside the cartilage over the 6-day period (FIG. 6A-6B and FIG. 16A-16D). Quantitative analysis of the fluorescence images revealed that PLL-PCL-doped TGFα-NPs exhibited more than a 4.76-fold improvement in cartilage penetration at day 6 compared with non-PLL-PCL doped TGFα-NPs (FIG. 6C). This result demonstrated the improved cartilage penetration and accumulation of TGFα-NPs with PLL-PCL.

Next, we directly injected TGFα-NPs or TGFα-DBCO labeled with IRDye 800CW into the knee joint to study their retention in the knee under healthy and OAs conditions (FIG. 6D). DMM was performed on the joints at 2 months before injection to mimic early OA stage. After a single injection, the fluorescence signal in the joints injected with TGFα-NPs was much higher than those injected with TGFα-DBCO at all time points, indicating the increased retention of TGFα-NPs (FIG. 6E). Quantitative analysis of fluorescence images showed that TGFα-NPs in OA joints were retained even longer than those in healthy joints (FIG. 6F).

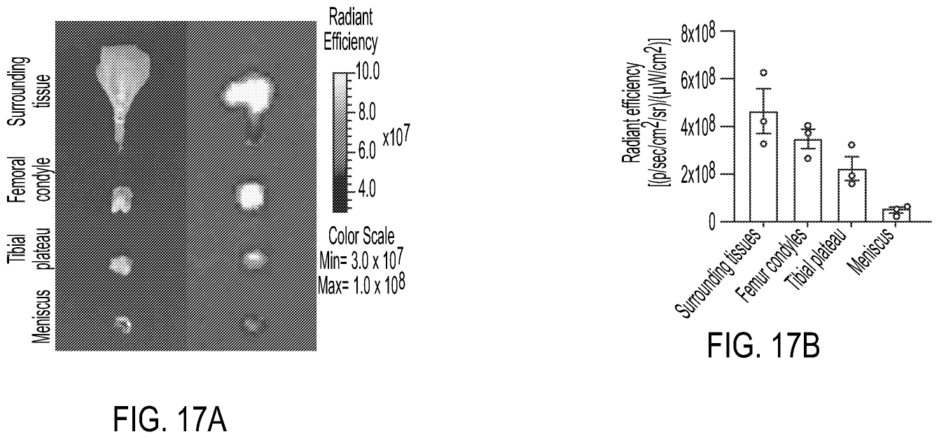

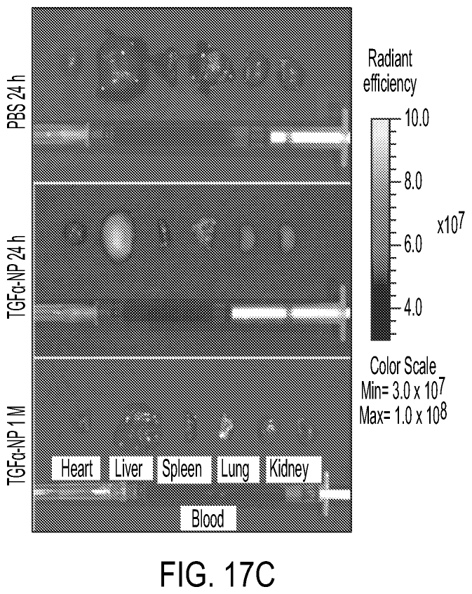

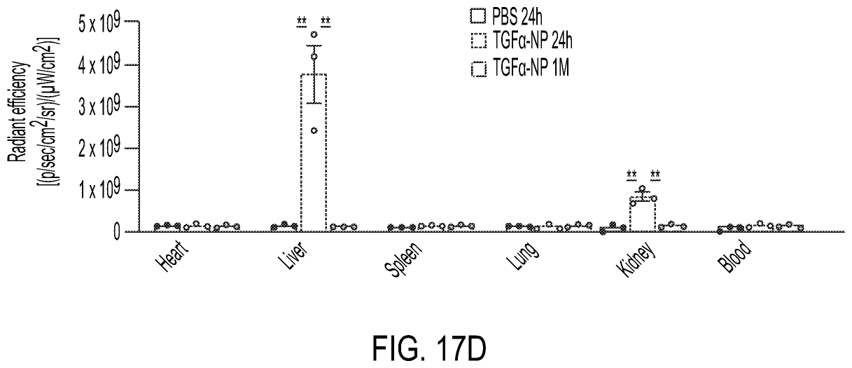

We also examined the biodistribution of TGFα-NPs in internal organs, blood, and joint components. At 24 hours post injection, fluorescent signals were detected on the cartilage surfaces of patellar, femur condyles and tibiae plateau as well as on meniscus (FIGS. 17A-17B). TGFα-NPs were mainly accumulated in liver and kidneys, but no signal was detected in the blood, heart and spleen, indicating that nanoparticles can be cleared quickly from circulation. One month later, there were no TGFα-NPs left in liver and kidney (FIGS. 17C-17D).

TGFα-NPs rescue OA cartilage from degeneration after DMM surgery

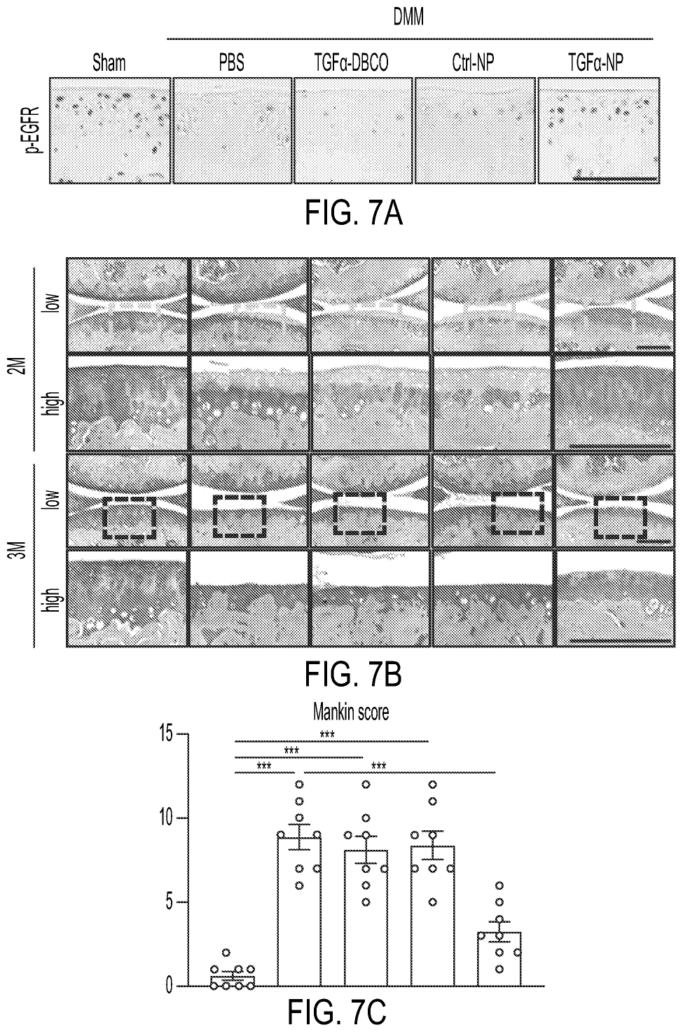

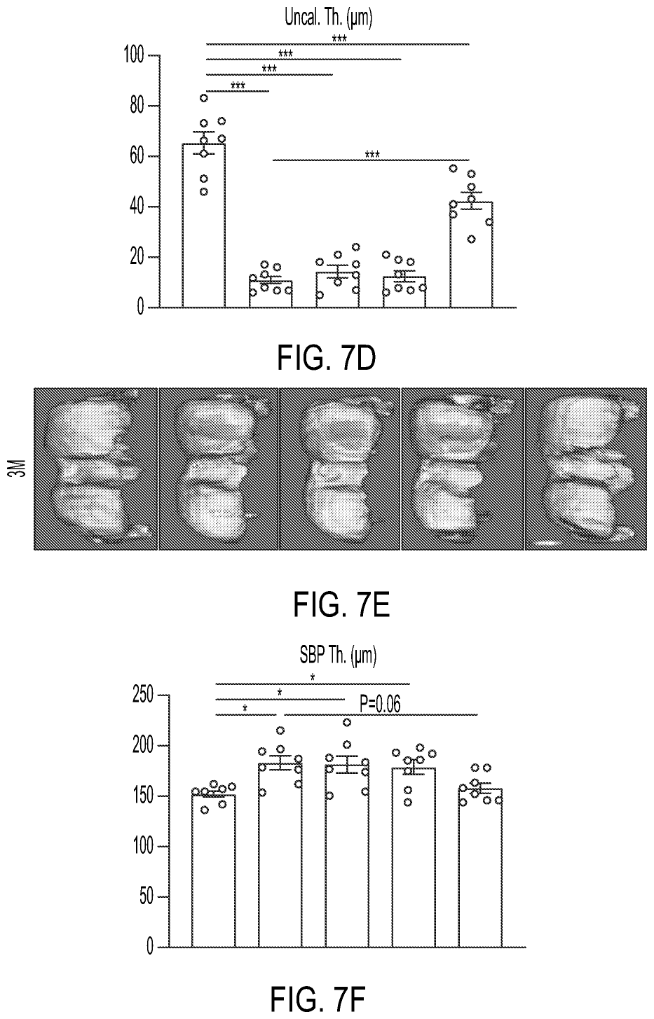

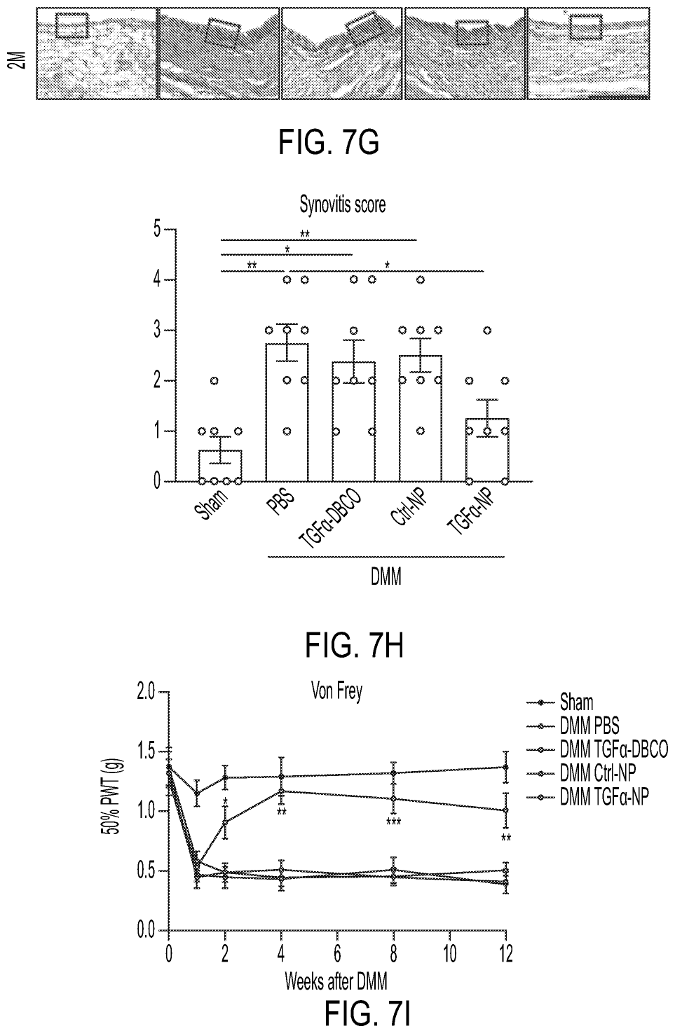

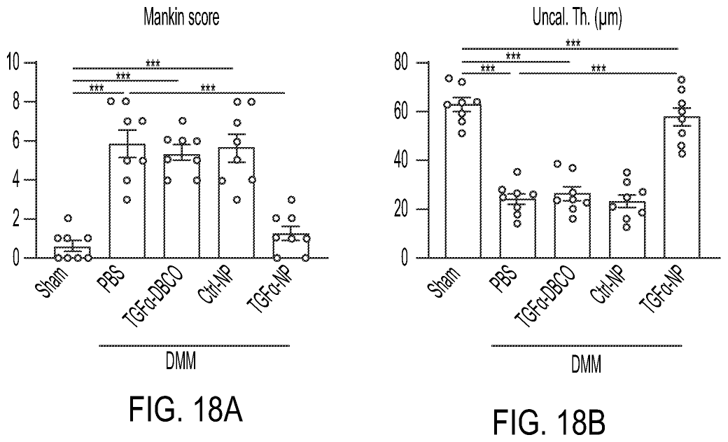

To test their therapeutic effect on OA, we injected TGFα-NPs into mouse knee joints after DMM once every 3 weeks. Control groups include knee joints injected with PBS, TGFα-DBCO, and Ctrl-NPs. In line with previous findings, EGFR activity, as indicated by p-EGFR, was decreased in articular cartilage after DMM. Injections of TGFα-NPs, but not TGFα-DBCO or Ctrl-NPs, successfully elevated cartilage EGFR activity to the level of the sham group (FIG. 7A). At 2 and 3 months post-surgery, both the TGFα-DBCO group and Ctrl-NP group displayed a similar pattern of cartilage degeneration, including erosion and surface fibrillation, similar to the PBS group (FIG. 7B). Mankin scores of these three control groups at 3 months post DMM were similarly around 8.5 (FIG. 7C), mainly due to the reduction of uncalcified cartilage thickness (FIG. 7D). Strikingly, knee joints in the TGFα-NP group maintained the cartilage integrity at 2 months after DMM and only displayed minor signs of degeneration at 3 months post DMM (FIG. 7B). The Mankin scores at both 2 and 3 months were drastically decreased compared to control groups and their uncalcified zones were mostly preserved (FIGS. 7C-7D and FIG. 18).

Subchondral bone sclerosis is a late OA symptom. Our previous study established a three-dimensional computed tomography (3D CT) approach to accurately measure the thickness of subchondral bone plate (SBP). Using this method, we confirmed that SBP thicknesses were significantly elevated in PBS, TGFα-DBCO, and Ctrl-NP-treated DMM knees (FIGS. 7E-7F). However, this increase was abolished in TGFα-NP-treated DMM knees. Synovitis is another sign of OA. We observed significant thickening of the synovial lining layer and increased synovitis scores in DMM knees with PBS, TGFα-DBCO, or Ctrl-NP treatment but not with TGFα-NP treatment (FIGS. 7G-7H). Moreover, von Frey assay revealed that TGFα-NP treatment attenuated OA-induced pain starting from 2 weeks post-surgery (FIG. 7I). Taken together, these results clearly demonstrate a therapeutic effect of intra-articular delivery of a novel EGFR ligand conjugated polymeric micellar nanoparticle.

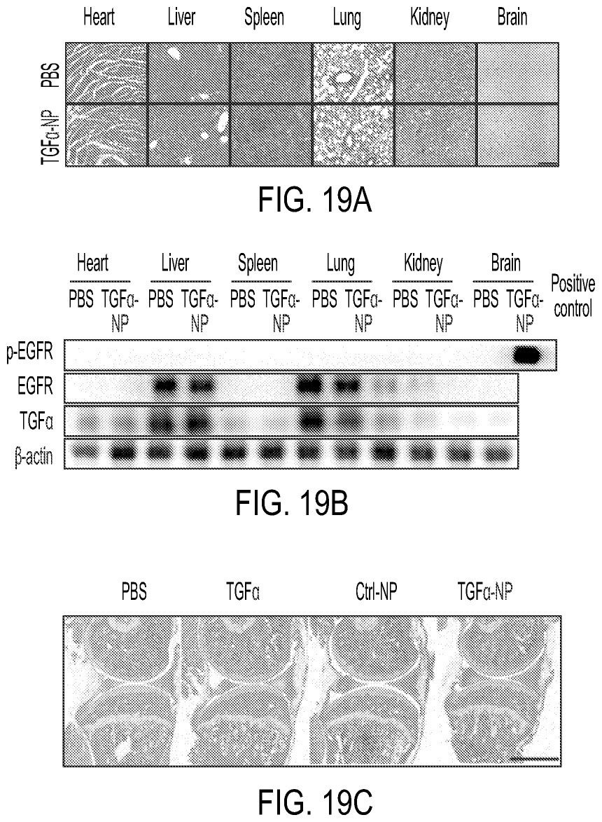

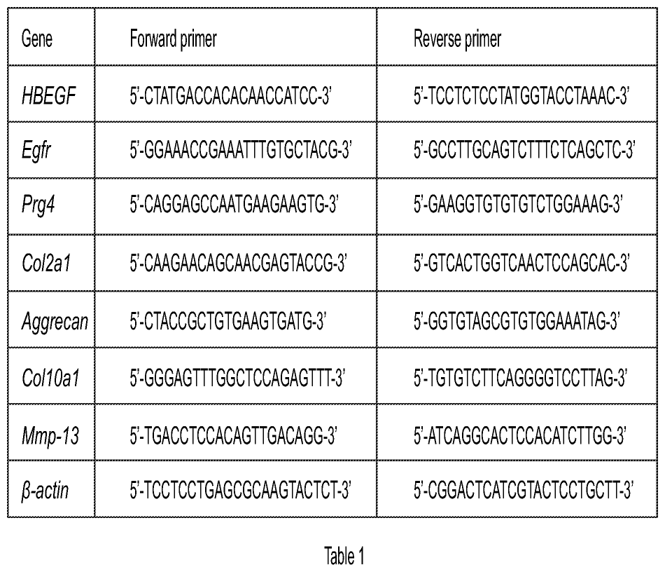

Lastly, we examined whether 2 months of intra-articular injections of TGFα-NPs caused any side effects on several major internal organs and overall joint structure. As shown in FIG. 19A, we did not detect any obvious morphologic changes in heart, liver, spleen, lung, kidney, and brain between PBS and TGFα-NP-treated mice. Western blots indicated no significant increase in EGFR activity in those organs after TGFα-NP injections (FIG. 19B). Liver and lung had the highest expression of EGFR and TGFα, which were not affected by TGFα-NP injections. Furthermore, the gross morphology of knee joints was not altered by 2 months of TGFα-NP treatment (FIG. 19C).

Discussion

Previous studies have demonstrated the important role of EGFR signaling in the development of articular cartilage and OA progression. While there are no doubts that EGFR signaling can execute both anabolic and catabolic actions on cartilage chondrocytes, differences exist regarding how this signaling pathway may be best modulated for OA treatment. In the present study, we first provide genetic evidence demonstrating that overactivation of EGFR signaling modestly thickens the articular cartilage and completely blocks OA progression after DMM surgery. Other joint tissues, such as bone, synovium, and meniscus appeared normal in mice up to 7 months of age and showed no pathological OA changes, such as osteophytosis and subchondral bone sclerosis, suggesting that EGFR signaling could be precisely applied in vivo to fulfill its anabolic actions without inciting catabolic effects. We also provide evidence using an advanced TGFα nanoparticle delivery system into knee joints that prevented DMM-induced OA initiation.

Currently, there are no disease-modifying drugs clinically approved for treating OA. Nonsteroidal anti-inflammatory agents (NSAIDs) have often been used for the short-term management of the pain symptoms in OA. More recently, several protein therapies, such as insulin-like growth factor (IGF) and fibroblast growth factor 18 (FGF-18), have shown promise for OA treatment. However, intra-articular delivery of these therapeutic proteins has been largely limited by their rapid clearance from the joint space and their low penetration into the dense, avascular cartilage matrix. Consistent with this, we also observed that intra-articular injection of free TGFα has low joint retention and poor cartilage penetration, and thus was ineffective in preventing OA development and progression. Due to its favorable pharmacokinetics, biodistribution, and specificity, nanoparticle-based drug delivery systems have been explored to improve drug delivery and therapeutic efficacy in OA treatment. For example, Geiger et al. developed dendrimer-based nanocarriers to deliver IGF-1 to chondrocytes within joint cartilage. The dendrimer-IGF-1 could penetrate full-thickness bovine cartilage and enhance the efficacy of IGF-1 in protecting both cartilage and bone in a rat surgical model of OA. Yan et al. used nanoparticle-based siRNA delivery to attenuate early inflammation in OA development.

We have addressed the TGFα delivery challenges by conjugating TGFα onto nanometer-sized polymeric micellar nanoparticles. Polymeric micellar nanoparticles are nanoscopic core/shell structures formed by amphiphilic block copolymers. Compared to other drug nanocarriers, the polymeric nanoparticles provide several clear advantages, including their relatively small size and the use of similar formulations in different preclinical and clinical studies. In this work, polymeric micellar nanoparticles were prepared from biocompatible and biodegradable polymers including PEG-PCL, PLL-PCL and pegylated phospholipids. PEG, PCL, PLL and phospholipids are clinically tested materials with well-characterized safety profiles. Moreover, the manufacture of these nanoparticles is simple, reproducible and scalable, which allows fast translation into clinic use.

We further utilized proximity-based sortase ligation to enable the highly efficient, site-specific bioconjugation of TGFα onto our nanoparticles. Currently, one of the greatest challenges faced when combining protein-based targeting ligands with nanoparticles is overcoming the low efficiency of bioconjugation. To address this limitation, we used a new bioconjugation strategy that utilizes a unique sortase fusion protein for the efficient and site-specific modification of the C-terminus of recombinant proteins. Based on this method, a DBCO moiety was ligated to the C-terminus of TGFα. The availability of the DBCO group, subsequently, allows for the facile bioconjugation of the TGFα to azide-labeled nanoparticles using highly efficient click chemistry. Finally, the TGFα-NPs exhibit therapeutic efficacy with no detectable side effects on joint structure and peripheral organs. The TGFα-NPs resolve the issues of short in vivo half-life and low cartilage penetration efficiency of free growth factors. Most notably, local delivery of TGFα-NPs into knee joints after OA injury effectively attenuates cartilage degeneration and blocks subchondral bone plate sclerosis and joint pain.

It is well-established that EGFR ligands, including TGFα and HBEGF, reduce anabolic gene expression (Sox9, Col2a1, and Aggrecan) and increase catabolic gene expression (MMP13) in cultured chondrocytes. Indeed, our studies using chondroprogenitors derived from HBEGF Over^{Col2} articular cartilage showed decreased anabolic gene expression and alcian blue staining when they undergo chondrogenic differentiation in vitro. However, these changes are relatively modest with a significant amount of cartilage matrix still remained in the pellet. Moreover, histology of adult HBEGF Over^{Col2} knees revealed no change in type II and type X collagen, and proteoglycan amounts, indicating that cell culture data might not be directly correlated to animal data. Interestingly, we also observed that MMP13 expression is decreased in HBEGF Over^{Col2} chondrogenic culture but not changed in the HBEGF Over^{Col2} joint. These data are contradictory to a previous report that adding HB-EGF to chondrocyte culture increases MMP13 expression.

Several EGFR activation mouse models have been investigated for OA study. Most of them used Mig6 knockout models, all with elevated EGFR activity in the cartilage. Unfortunately, the global ablation of Mig6 caused severe joint deformity in young mice. The cartilage-specific (Col2-Cre) and skeletal-specific (PrxI-Cre) knockout of Mig6 had a much minor joint phenotype, with initial anabolic expansion of articular cartilage followed by modest degeneration or osteophyte formation at a later age. A previous report took advantage of DTR to construct DermoI-Cre HBEGF (HBEGF OverDerm^{DermoI}) mice.

At a young age, these mice develop chondrodysplasia, chondroma, OA-like joint defects, as well as bone phenotypes. While DermoI-Cre broadly targets mesenchymal lineage cells, the Col2-Cre and Aggrecan-CreER used here are more specific for cartilage tissue. In contrast to HBEGF Over^{DermoI} mice, our HBEGF Over^{Col2} and Over^{AgcER} mice do not show any joint and bone abnormalities except anabolic expansion of cartilage. Therefore, we propose that the therapeutic effect of EGFR signaling depends on its activation level and specificity. Mig6 global knockout and HBEGF Over^{DermoI} mice possess the highest EGFR activity not only in cartilage but also in other organs, thus tipping the balance more towards catabolic actions on cartilage. Mig6 CKO mice have increased EGFR activity but not as high as the previous two models such that they exhibit anabolic actions first and then catabolic actions. In addition, we cannot rule out the possibility that Mig6 knockout mice have off-target effects because Mig6 also regulates signaling pathways other than EGFR, such as HGF/Met. Due to their cartilage- and EGFR-specificity, HBEGF Over^{Col2} and Over^{AgcER} mouse models, as well as joint delivery of TGFα-NPs, demonstrate that one can precisely control EGFR signaling for cartilage anabolic actions only without incurring undesired catabolic effects.

Another concern is based on the initial findings that TGFα and HBEGF levels are elevated in degenerated OA cartilage. Accordingly, it was proposed that EGFR inhibitors could be repurposed for OA treatment. To date, the results of EGFR inhibitors on rodent OA progression are mixed and often contradictory, depending on the gender and type of inhibitors used in experiments. In our hands, we constantly observed accelerated OA by Gefitinib treatment. Our previous study clearly showed that EGFR activity is most concentrated in the superficial layer of articular cartilage, which is drastically reduced during OA initiation. In this study, overactivation of EGFR in animal models, particularly using inducible Aggrecan-CreER, or by TGFα-NP injections demonstrates that elevating EGFR activation at the early stage of OA is beneficial. The underlying mechanisms, as shown here and in our previous studies, involve the protection of superficial layer from OA-induced destruction. At a later stage when the superficial zone is disrupted and the levels of TGFα and HBEGF are elevated, whether further increasing EGFR activity could still render a protective or even reparative effect on cartilage needs further investigation. During OA degeneration, new chondrocyte cell clusters are frequently formed under the damaged cartilage surface in an attempt to repair and regenerate. Those cell clusters are proliferative with stem cell properties. However, under normal circumstances, impaired cartilage does not repair by itself. Without being bound to any particular theory, one can speculate that as growth factors for mesenchymal progenitors, TGFα and HB-EGF are likely to be up-regulated for forming cell clusters after damage, yet their levels are not high enough to regenerate the cartilage. Therefore, exogenous EGFR ligand can be helpful in attenuating OA progression, even at a late stage.

There is a great unmet medical need for a disease-modifying OA drug. In this disclosure, we demonstrate the feasibility of targeting EGFR signaling to block OA initiation and constructed a drug for such treatment without obvious side effects.

Materials and Methods

Study design

This study was performed to evaluate whether activating EGFR signaling specifically in joint cartilage could protect articular cartilage from OA degeneration. This objective was addressed by (i) examining articular cartilage phenotype in HBEGF Over^{Col2} and Over^{AgcER} mice, (ii) delineating the cellular and molecular changes in their articular cartilage, (iii) characterizing their responses toward OA surgery, (iv) synthesizing and characterizing TGFα-NPs, and (v) studying the effect of intra-articular injection of TGFα-NPs on preventing OA progression. All data presented here have been replicated in independent cohorts of six or more mice or in three or more biological replicates for in vitro experiments. Samples were assigned randomly to the experimental groups. Data collection for each experiment is detailed in the respective figures, figure legends, and methods.

Animals

All animal work performed in this study was approved by the Institutional Animal Care and Use Committee (IACUC) at the University of Pennsylvania. In accordance with the standards for animal housing, mice were group housed at 23-25° C. with a 12 h light/dark cycle and allowed free access to water and standard laboratory pellets.

Col2-Cre mice or Aggrecan-CreER mice were bred with Rosa-DTR mice to generate Col2-Cre DTR (HBEGF Over^{Col2}) and Aggrecan-CreER DTR (HBEGF Over^{AgcER}) mice, respectively, and their WT (DTR or Cre only) siblings. All mouse lines were purchased from Jackson Laboratory (Bar Harbor, ME, USA).

To induce OA, male mice at 3 months of age were subjected to DMM surgery or sham surgery at right knees as described previously. Briefly, in DMM surgery, the joint capsule was opened immediately after anesthesia and the medial meniscotibial ligament was cut to destabilize the meniscus without damaging other tissues. In Sham surgery, the joint capsule was opened in the same fashion but without any further damage.

HBEGF Over^{AgcER} mice and WT controls received Tamoxifen injections (75 mg/kg/day) for 5 days before DMM surgery. For EdU incorporation study, 3-day-old mice received intraperitoneal injections of EdU (2.5 mg/kg) for 4 days. Knee joints were harvested on the 5th day or 3 weeks later.

For the treatment study, male C57Bl/6 mice (Jackson Laboratory) were randomly divided into 5 groups: sham surgery (sham), DMM and PBS treatment, DMM and free TGFα-DBCO treatment (TGFα-DBCO), DMM and Ctrl-NP treatment (Ctrl-NP), and DMM and TGFα-NP treatment (TGFα-NP). Treatments were given by intra-articular injection of 10 μl of PBS, TGFα-DBCO (10 μM TGFα content), Ctrl-NPs (0 μM TGFα content), or TGFα-NPs (10 μM TGFα content) once every three weeks starting from right after DMM surgery. A total number of 3 injections were applied to 2 months post-surgery group and 4 injections were applied to 3 months post-surgery group.

TGFα-NP synthesis

Synthesis of azido-terminated Poly(ε-caprolactone) (PCL-N₃)

PCL-OH (1.6 g, 0.40 mmol, Mw 4000) was dissolved in anhydrous chloroform (15 mL) followed by addition of TEA (202 mg, 2 mmol). The mixture was then added to a solution of MsCl (229 mg, 2 mmol) in chloroform (3 mL) at 0° C. under N₂ stream. The reaction was carried out overnight, under stirring at room temperature. After the reaction, the polymer was recovered as a white solid by precipitating into ethyl ether and vacuum-drying. This mesylated copolymer (1.08 g, 0.27 mmol) was dissolved in DMF (12 mL), and reacted with sodium azide (800 mg, 12.30 mmol) at 45° C. under stirring for 3 days. After the reaction, DMF was evaporated and the concentrate was diluted with chloroform (40 mL), and then washed five times with water and brine. The organic layer was dried over MgSO₄, filtered, concentrated, and then precipitated into ethyl ether (0.97 g, 90%).

Synthesis of copolymers poly(c-caprolactone)-block-poly(L-lysine) (PCL4K-b-PLL3.3K)

The PCL-b-PLL was synthesized by click reaction between PCL-N3 (Mw: 4000) and propargyl-PLL (Mw: 3300). Briefly, PCL-N3 (60 mg, 0.015 mmol), propargyl-PLL (55 mg, 0.0167 mmol), CuSO4 (0.375 mg, 0.0015 mmol), sodium ascorbate (0.594 mg, 0.003 mmol) and 10 mL degassed DMF were added into a 30 mL Schlenk flask under a nitrogen atmosphere. The flask was sealed and placed into an oil bath. The reaction was carried out at 45° C. with magnetic stirring for 3 days, and the mixture was dialyzed against water to remove the residual propargyl-PLL. The resulting copolymer was lyophilized to get the powder.

Synthesis of TGFα-GGG and TGFα-DBCO

The human TGFα gene sequence (50 aa) was ordered from Integrated DNA Technologies (IDT) and cloned into the Sortase-Tag Expressed Protein Ligation (STEPL) system. Briefly, TGFα was fused in series with the sortase A (Srt A) substrate sequence (LPXTG), SrtA enzyme, and a His12-tag. The sequence-confirmed plasmid construct was heat-shot transformed into T7 express competent cells (NEB). On the next day, colonies were cultured in autoinduction medium (Formedium, UK) with 100 ug/ml Ampicillin (Corning) and were shaken at 150 rpm at 25° C. for 2 days. Afterwards, the cultures were pelleted by centrifugation at 5000×g for 15 mins and the cells were lysed with 1% (g/v) octylthioglucoside (OTG, GoldBio) in PBS, with protease inhibitor (Thermo Fisher). The lysate was centrifuged again at maximum speed for 20 minutes and then loaded into a cobalt resin (Thermo Fisher) for capturing the TGFα fusion protein. After washing with 10mM Imidazole and 1xPBS buffer, the resin was incubated with 1×PBS+50 μM CaCl₂+2 mM GGG (Santa Cruz Biotechnology) at 37° C. for 1 hour or 1×PBS+50 μM CaCl₂+200 μM GGGSK-DBCO (LifeTein) at 37° C. for 4 hours. The excess GGG or GGGSK-DBCO was removed by spin filter (Amicon Ultra-4, 3000 MWCO). The TGFα concentration was quantified by BCA assay according to the manufacturer's instructions.

Synthesis of fluorescent labeled TGFα

We synthesized rhodamine-TGFα for penetration assay. Rhodamine-TGFα was prepared using a molar ratio of 1/10 of NHS-rhodamine (Thermo Scientific) /TGFα-DBCO. Specifically, 1.5 mL 60 μM TGFα-DBCO (in 0.1 M PBS) was mixed with 18 μL 50 μM NHS-Rhodamine (in DMF). After shaking at room temperature for 2h, unconjugated NHS-Rhodamine was removed by centrifugal filter devices (Amicon Ultra-4, 3000 MWCO, Millipore Corp.).

IRDye 800CW-labeled TGFα was prepared for retention assay. It was synthesized utilizing a molar ratio of 1/10 of IRDye 800CW NHS Ester (LI-COR, Inc)/TGFα-DBCO. Specifically, 200 μL 85 μM TGFα-DBCO (in 0.1 M PBS) was mixed with 17 μL 10 mM IRDye 800CW NHS Ester (in DMSO). After shaking at room temperature for 2h, unconjugated IRDye 800CW NHS Ester was removed by centrifugal filter devices (Amicon Ultra-4, 3000 MWCO, Millipore Corp.).

Synthesis of TGFα-conjugated nanoparticles

TGFα-conjugated nanoparticles (TGFα-NPs) were prepared via click reaction. Briefly, stock solutions of poly(ethylene glycol) (4000)- polycaprolactone (3000) copolymer (denoted PEG-PCL, Polymer Source, Canada), polylysine (3300)-polycaprolactone (4000) copolymer (denoted PLL-PCL) and 1,2-distearoyl-sn-glycero-3-phosphoethanolamine-N -[azido(polyethylene glycol)-5000] (ammonium salt) (denoted DSPE-PEG5K-N3, Avanti Polar Lipids, Inc) in chloroform were mixed in the following molar ratios: PEG-PCL/PLL-PCL/DSPE-PEG5K-N3 (55/20/25). The total amount of PEG-PCL for each of the nanoparticle compositions was 1 mg. For non PLL-PCL doped nanoparticles, 75 mol % PEG-PCL/25 mol % DSPE-PEG5K-N3 was used. The chloroform was removed using a direct stream of nitrogen prior to vacuum desiccation for overnight. Nanoparticles were formed by adding an aqueous solution (0.1 M PBS, pH 7.4) to the dried film and incubating in a 60° C. water bath for 3 minutes and then sonicating for another 3 minutes at the same temperature. Samples were filtered through a 0.22 μm cellulose acetate membrane filter (Nalgene, Thermo Scientific) and stored in the dark at 4° C.

To prepare TGFα-NPs, azide-modified nanoparticles were mixed with TGFα-DBCO at a molar ratio of 1 to 1 in 0.1 M PBS (pH 7.4). Reactions were mixed overnight at room temperature and then purified by centrifugal filter devices (Amicon Ultra-4, 50K MWCO, Millipore Corp.). Similar methods were used to prepare Rhodamine or IRDye 800CW-labeled TGFα-NPs. The diameter and size distribution of the nanoparticles were measured with dynamic light scattering (DLS, Malvern, Zetasizer, Nano-ZS). Zeta potential was also determined by Zetasizer Nano analyzer (Malvern, UK). The morphology of the nanoparticles was characterized by transmission electron microscope (TEM) (JOEL 1010) using a negative-staining technique (i.e. phosphotungstic acid). Fluorescence spectra measurements were made on a SPEX FluoroMax-3 spectrofluorometer (Horiba Jobin Yvon).

TGFα-NP characterization

Stability study

For stability assay, TGFα-NPs were stored in 0.1×PBS at 4° C. Measurement of nanoparticle structural integrity was acquired by monitoring the hydrodynamic diameter over the course of one week by DLS. In addition, the in vitro stability of TGFα-NPs was also measured by DLS in 50% bovine synovial fluid (Vendors, Lampire biological laboratories) at 37° C. for 24 hours. TGFα-NP stability was tested in triplicate.

Cell viability study

For cell viability assay, we used primary mouse chondrocytes isolated from the distal femoral and proximal tibial epiphysis of mice (3-6 days old) via enzymatic digestion as described previously. Cells (5000/well) were seeded in 96-well plates and incubated overnight. The diluted TGFα-NPs were added to wells at five different concentrations ranging from 10 μM to 0.3125 μM (10, 5, 2.5, 1.25, 0.625, 0.3125 μM). After 24 h incubation, the cells grew in 100 μL of fresh DMEM/F12 medium with 10 μL of MTT assay stock solution added to each well and incubated for 4 h. The formazan was dissolved by adding 100 μL of detergent to each well and then incubated for another 4 h. Finally, the absorbance of formazan product was measured on a Tecan microplate reader (BioTek Instruments, Inc.) at 570 nm. Cell viability was calculated using the following equation:

$$Cell viability ( \% ) = \frac{{Absorbance}_{sample}}{{Absorbance}_{control}} \times 100$$

TGFα-NP activity study

Primary chondrocytes were seed in 6-well plates and reached to 80% confluency. Cells were then incubated in fresh medium containing TGFα (0 or 15 ng/ml TGFα content), Ctrl-NPs (i.e. nanoparticles without TGFα), TGFα-NPs (15 or 100 ng/ml TGFα content) for 15 minutes at 37° C. The cells were washed twice with PBS and then lysed with lysis buffer for Western blot analysis. Primary chondrocytes seeded in 6-well plate were also incubated in fresh medium with or without TGFα-NPs (15 ng/ml TGFα content) for 48 hours. Cell morphology was observed under bright field of inverted microscope.

Cell binding study

Primary chondrocytes plated in 24-well plates were washed once with PBS and then incubated in fresh media containing TGFα-NPs (10 nM TGFα content) for 2 hours. For competitive inhibition experiments, cells were treated with the same amount of TGFα-NPs but in the presence of 100 μg/ml free TGFα in the media. Prior to acquisition of fluorescence images, cells were washed with PBS two times, and then fixed and mounted with DAPI Fluoromount-G Mounting Medium. Confocal images were taken using confocal microscope (Zeiss LSM 710).

Cartilage explant penetration and uptake study

For bovine cartilage explant penetration assay, we obtained young (1-2 weeks old) bovine knee join IS from Lampire biological laboratories, harvested cartilage explants from the trochlear groove using biopsy punch (6 mm in diameter and 2 mm in thickness), cultured them in chemically defined medium (DMEM, 1% ITS+Premix, 50 μg/ml L-protine, 0.1 μM dexamethasone, 0.9 nM sodium pyruvate and 50 μg/ml ascorbate 2-phosphate) in 48-well plate. The cartilage explants were then incubated with rhodamine labeled-TGFα-DBCO, TGFα-NPs (without PLL-PCL) or TGFα-NPs (with PLL-PCL) in 500 μl of culture medium for 48, 96 or 144 hours at 37° C. under gentle agitation with medium replacement every other day. In all cases, the final rhodamine concentration in the culture medium was 10 μM. After incubation, cartilage explants were washed three times with PBS, fixed with 4% PFA (Paraformaldehyde), dehydrated with 20% sucrose+2% PVP (Polyvinylpyrrolidone) followed by embedding with 20% sucrose+2% PVP+8% gelatin. Sections were mounted with DAPI Fluoromount-G Mounting Medium on glass slides and immediately observed under confocal microscope (Zeiss LSM 710). All images are taken under the same laser power, intensity and offset. Quantitative analysis was performed on maximum intensity projections of Z-stack images taken from 100 μm thick sections.

For bovine cartilage explant uptake assay, 300 μl of IRDye 800CW-labeled TGFα-DBCO, TGFα-NPs (without PLL-PCL) or TGFα-NPs (with PLL-PCL) was added to bovine cartilage explants. The final IRDye 800CW concentration in the culture medium was 10 uM. The explants were incubated for 48 hours at 37° C. and 5% CO₂ under gentle agitation. The explants were then removed from the medium, washed tree times with PBS, imaged by IVIS (Spectrum, PerkinElmer). All images are taken under the same laser power, intensity and offset. Radiant efficiency it a fixed anatomical region of interest (ROI) was measured using Living image software.

Intra-articular retention and systemic biodistribution study

For in vivo retention assay, we injected 10 μl of 10 μM IRDye 800CW-labeled TGFα-DBCO or TGFα-NPs (with PLL-PCL) in healthy and OA (8 weeks post DMM surgery) mouse knees (3 months old). An IVIS (Spectrum, PerkinElmer) was used to serially acquire fluorescence images within each joint over a period of 4 weeks. All images are taken under the same laser power, intensity and offset. Using Living Image software, radiant efficiency within a fixed anatomical region of interest (ROI) was measured.

For in vivo biodistribution assay, we injected 10 μl of PBS or 10 μM IRDye 800CW-labeled TGFα-NPs in mouse knees (3 months old). At 24 hours or 1 month after injection, the mice were sacrificed and the knee joints, blood, and major organs (heart, liver, spleen, lung, kidney) were harvested. Knees were dissected to isolate the major joint components, including the surrounding tissues (quadriceps, patella, patellar ligament, synovium, fat pad), femoral condyles, tibial plateau and meniscus. All the major joint components, blood and organs were imaged using the IVIS under the same laser power, intensity and offset. The data was analyzed as described above.

Micro-computed tomography (microCT) analysis

After euthanasia, mouse knee joints were harvested, fixed in 4% paraformaldehyde for 2 days, rinsed with running water, and stored in 1×PBS. A 3-mm region from the distal femur and the proximal tibia were then scanned at a 6-μm isotropic voxel size with a microCT 35 scanner (Scanco Medical AG, Bruttisellen, Switzerland). All images were smoothened by a Gaussian filter (sigma=1.2, support=2.0).

SBP thickness was calculated as previously described. Briefly, sagittal images were contoured for the SBP followed by generating a 3D color map of thickness for the entire SBP. This map was converted to a grayscale thickness map, whose histogram was then used for the quantification of the average SBP thickness at any defined area.

To analyze metaphyseal trabecular bone, the proximal tibiae were scanned at a 6-μm isotropic voxel size. Images from 0.6-1.8 mm below growth plate were thresholded corresponding to 472.1 mg HA/cm3 and contoured for trabecular bone. Bone volume fraction (BV/TV), trabecular thickness (Tb.Th), trabecular separation (Tb.Sp), trabecular number (Tb.N), and structure model index (SMI), bone mineral density (BMD) were calculated by 3D standard microstructural analysis.

Histology

After euthanasia, mouse knee joints were harvested and fixed in 4% paraformaldehyde overnight followed by decalcification in 0.5 M EDTA (pH 7.4) for 4 weeks prior to paraffin embedding. A serial of 6 μm-thick sagittal sections (about 100) were cut across the entire medial compartment of the joint until ACL junction. To measure the thicknesses of articular cartilage, chondrocyte numbers and growth plate thickness, 3 sections from each knee, corresponding to 1/4 (sections 20-30), 2/4 (sections 45-55), and 3/4 (sections 70-80) regions of the entire section set, were stained with Safranin O/Fast green or hematoxylin and eosin (H&E) and quantified using BIOQUANT software. The final measurement is an average of these three sections. We defined uncalcified cartilage as the area from articular surface to tide mark and calcified cartilage as the area from tide mark to cement line. A similar approach was used on H&E stained sections to count the number of superficial chondrocytes (flat cells at the cartilage surface) and to measure synovial inflammation score as defined previously. The method to measure Mankin Score was described previously. Briefly, two sections within every consecutive six sections in the entire section set for each knee were stained with Safranin O/Fast green and scored by two blinded observers. Each knee received a single score representing the maximal score of all its sections.

Paraffin sections were used for immunohistochemistry. After appropriate antigen retrieval, slides were incubated with primary antibodies, such as rabbit anti-EGFR (CST, 4267), rabbit anti p-EGFR (Abcam, ab40815), rabbit anti-p-ERK (CST, 4370), rabbit anti-ERK (CST, 4695), rabbit anti-Ki67 (Abcam, ab15580), rabbit anti-HBEGF/DTR (Abcam, ab192545), rabbit anti-TGFα (Abcam, ab9585), PRG4 (Abcam, ab28484) at 4° C. overnight, followed by binding with biotinylated secondary antibodies and DAB color development. The terminal deoxynucleotidyl transferase dUTP nick end labeling (TUNEL) assay was carried out according to the manufacturer's instructions (Millipore, s7101). Mouse tissues, such as liver, spleen, lung etc, were also collected for paraffin sections followed by H&E staining to observe their morphology.

For EdU labeling experiment, joints were harvested and fixed in 4% paraformaldehyde overnight followed by decalcification in 0.5 M EDTA (pH 7.4) for 1 week prior to OCT embedding. Using a similar approach as described above, 3 representative sections were selected for EdU staining according to the manufacturer's instructions (Invitrogen, C10337). Positive cells within periarticular region or articular cartilage region were quantified using ImageJ software.

AFM-nanoindentation

Nanoindentation was performed on femoral articular cartilage surface as we previously described. Freshly dissected femoral condyle cartilage was indented at more than 10 locations by a borosilicate colloidal spherical tip (R ≈5 μm, nominal spring constant k=7.4 N/m, AIO-TL tip C, NanoAndMore) with maximum indentation depth of ˜1 μm at 10 μm/s indentation rate using a Dimension Icon AFM (BrukerNano) in PBS with protease inhibitors. The effective indentation modulus, E_{ind} (MPa), was calculated by fitting the whole loading portion of each indentation force-depth curve using the Hertz model.

OA pain analysis

The knee joint pain after DMM surgery was evaluated in mice at 1 month after surgery using von Frey filaments as described previously. An individual mouse was placed on a wire-mesh platform (Excellent Technology Co.) under a 4×3×7 cm cage to restrict their move. Mice were trained to be accustomed to this condition every day starting from 7 days before the test. During the test, a set of von Frey fibers (Stoelting Touch Test Sensory Evaluator Kit #2 to #9; ranging from 0.015 to 1.3 g force) were applied to the plantar surface of the hind paw until the fibers bowed, and then held for 3 seconds. The threshold force required to elicit withdrawal of the paw (median 50% withdrawal) was determined five times on each hind paw with sequential measurements separated by at least 5 min.

Chondrocyte culture, immunoblotting, and real time RT-PCR analysis

Adult chondroprogenitors were harvested from articular cartilage of 5-month-old mouse knee joints. Briefly, cartilage was peeled off from femoral condyles and tibial plateau by sterile scalpel under dissection microscope and incubated in 0.25% trypsin (Invitrogen) for 1 h, followed by 2 h digestion with 900 U/ml type I collagenase (Worthington Biochemical). Dissociated cells from the second digestion were cultured in DMEM medium containing 10% fetal bovine serum (FBS), 100 μg/ml streptomycin and 100 U/ml penicillin. For CFU-F assay, 1×10⁴ cells were seed in 6-well plate and cultured for 7 days followed by crystal violate staining. CFU number was counted under microscope. For proliferation assay, cells were seeded in culture medium. Cell counting was performed on the indicated days. For apoptosis assay, cells at 40-60% confluency were serum starved overnight and then pretreated with either vehicle or 25 ng/ml tumor necrosis factor a (TNFα) (Pepro-Tech). Two days later, apoptotic cells were quantified using ethidium bromide (5 mg/ml) and acridine orange (5 mg/ml) staining as described previously. For chondrogenic differentiation assay, confluent cells were cultured in differentiation medium (growth medium with 50 μg/ml L-ascorbic acid). Media were changed twice a week.

To perform Western blot, cell lysate was solubilized in RIPA buffer (50 mM Tris, pH 7.4, 100 mM NaCl, 1% sodium deoxycholate, 1% Triton-X 100, and 0.1% SDS) with protease inhibitor. Cell lysate (50 mg) was separated by SDS—PAGE and transferred onto PVDF membrane. Immunoreactive protein bands were visualized using rabbit anti-EGFR (CST, 4267), rabbit anti p-EGFR (Abcam, ab40815), rabbit anti-p-ERK (CST, 4370), rabbit anti-ERK (CST, 4695), rabbit anti-HBEGF/DTR (Abcam, ab192545), rabbit anti-TGFα (Abcam, ab9585), (3-actin (CST, 4970) and corresponding secondary antibodies, followed by chemiluminescence (Amersham ECLTM Western Blotting Detection Reagents, GE healthcare).

RNA was harvested from chondrocyte culture using Tri Reagent (Sigma). Taqman® Reverse Transcription kit (Applied BioSystems) was used to reverse transcribe mRNA into cDNA. Following this, PCR was performed using a Power SYBR® Green PCR Master Mix kit (Applied BioSystems). The primer sequences for the genes used in this study are listed in Supplementary Table 1.

Statistical analysis

Data are expressed as means ±standard error of the mean (SEM) and analyzed by t-tests, one-way ANOVA with Dunnett's or Turkey's posttest and two-way ANOVA with bonferroni's or Turkey's post-test for multiple comparisons using Prism 8 software (GraphPad Software, San Diego, CA). For cell culture experiments, observations were repeated independently at least three times with a similar conclusion, and only data from a representative experiment are presented. Values of p<0.05 were considered significant.

Figures

FIGS. 1A-1G illustrate that the overexpression of HBEGF in chondrocytes expands mouse growth plate and articular cartilage without affecting the gross appearance of knee joints. (FIG. 1A) Western blot results reveal increased protein levels of HBEGF and EGFR downstream signals (p-EGFR and p-ERK) in articular cartilage chondrocytes derived from HBEGF OverCol2 mice. (FIG. 1B) Safranin O/Fast Green staining of knee joints from 5-month-old mice shows no abnormalities in HBEGF OverCol2 mice compared with their control littermates. Scale bar, 1 mm. (FIG. 1C) Safranin O/Fast Green staining of tibial growth plate in WT and HBEGF OverCol2 mice at 1 and 5 months of age. Scale bar, 200 μm. (FIG. 1D) The thicknesses (Th.) of proliferative zone (PZ) and hypertrophic zone (HZ) in the growth plate of 1-month-old mice were quantified. n=5 mice/group. (FIG. 1E) The growth plate thickness (GP Th.) was quantified in 1- and 5-month-old mice. n=5 mice/group. (FIG. 1F) Safranin O/Fast Green staining of articular cartilage in WT and HBEGF OverCol2 mice at 1 and 5 months of age. Scale bar, 200 μm. (FIG. 1G) Average thicknesses of uncalcified zone (Uncal. Th.), calcified zone (Cal. Th.), and total tibial articular cartilage were quantified in 1- and 5-month-old mice. n=8 mice/group. Statistical analysis was performed using two-way ANOVA with Bonferroni's post hoc analysis. Data presented as means ±SEM. *p<0.05, **p<0.01, ***p<0.001.

FIGS. 2A-2K illustrate the overexpression of HBEGF increases chondroprogenitors in articular cartilage. (FIG. 2A) H&E staining of femoral articular cartilage in WT and HBEGF Over' mice at 1 and 5 months of age. Scale bar, 50 μm. (FIG. 2B) Chondrocyte numbers in superficial zone (SZ), transition and middle zones (TZ+MZ), calcified zone (CZ), and entire femoral articular cartilage were quantified at 1 and 5 months of age. n=8 mice/group. (FIG. 2C) Immunostaining of Ki67, TUNEL, and Prg4 in tibial articular cartilage of 5-month-old WT and HBEGF Over^{Col2} mouse. (FIG. 2D) The percentages of Ki67⁺, TUNEL⁺, and Prg4⁺cells within articular cartilage were quantified. n=8 mice/group. (FIG. 2E) Long term EdU labeling reveals more slow cycling cells in the tibial articular cartilage of HBEGF Over^{Col2} mice. Mice received daily EdU injections from P4-6 and their joints were harvested at 1 and 4 weeks of age for EdU staining. Dashed lines outline periarticular layer (1 week of age) and articular cartilage (4 weeks of age) for analysis. Scale bar, 100 μm. (FIG. 2F) Quantification of EdU⁺ cells in outlined regions. n=5 mice/group. (FIG. 2G) CFU-F assay using chondrocytes dissociated from mouse knee joints at 5 months of age shows more progenitors in HBEGF Over^{Col2} mice compared to WT mice. Scale bar, 0.5 cm. (FIG. 2H) Quantification of CFU-F number in 1×10⁴ cells. n=5 independent experiments. (FIG. 2I) Primary chondroprogenitors from 5-month-old HBEGF Over^{Col2} knee joints proliferate faster than those from WTjoints. Cells were seeded at the same density on day 0 and their numbers were counted every other day. n=5 independent experiments. (FIG. 2J) Apoptosis assay of primary chondrocytes from 5-month-old WT and HBEGF Over^{Col2} knee joints. Cells were incubated with or without TNFα (25 ng/ml) for 2 days before analysis. n=5 independent experiments. (FIG. 2K) qRT-PCR analyzes the relative gene expression in chondroprogenitors from WT and HBEGF Over^{Col2} knee joints undergoing 2 weeks of chondrogenic differentiation. n=3 independent experiments. (FIG. 2L) Alcian blue staining are performed on the same cells as above. Scale bar, 200 μm. Statistical analysis was performed using two-way ANOVA with Turkey's post hoc analysis for (B), (F) and (J) and paired t-test for (FIG. 2D), (FIG. 2H), (FIG. 2I) and (FIG. 2K). Data presented as means ±SEM. *p<0.05, **p<0.01, ***p<0.001.

FIGS. 3A-3G illustrate that overexpressing HBEGF in articular cartilage delays OA progression. (FIG. 3A) Schematic graph shows the study protocol of WT and HBEGF Over^{Col2} mice with DMM surgery. (FIG. 3B) Safranin O/Fast Green staining of WT and HBEGF Over^{Col2} DMM and sham joints at the medial site at 5 and 7 months of age. The bottom panel shows magnified images of cartilage damage sites (yellow boxed area) from the middle panel. Scale bars, 200 μm. (FIG. 3C) The OA severity was measured by Mankin score. n=8 mice/group. (FIG. 3D)Schematic graph shows the study protocol of WT and HBEGF Over^{AgcER} mice with Tamoxifen injections and DMM surgery. (FIG. 3E) Safranin O/Fast Green staining of WT and HBEGF Over^{AgcER} DMM and sham joints at the medial site at 7 months of age. The bottom panel shows magnified images of cartilage damage sites (yellow boxed area) from the middle panel. Scale bars, 200 μm. (FIG. 3F) The OA severity was measured by Mankin score. n=8 mice/group. (FIG. 3G) Nanoindentation assay was performed on femoral cartilage surface at 1 month post-surgery. End, modulus. n=4-5 mice/group. Statistical analysis was performed using two-way ANOVA with Turkey's post hoc analysis. Data presented as means ±SEM. *p<0.05, **p<0.01, ***p<0.001.

FIGS. 4A-4D illustrate that the protective action of HBEGF overexpression on articular cartilage during OA development is EGFR-dependent. (FIG. 4A) Safranin O/Fast Green staining of vehicle- and Gefinitib-treated WT and HBEGF Over^{AgcER} knee joints at the medial site at 2 months post-surgery. The bottom panel shows magnified images of cartilage damage sites (yellow boxed area) from the middle panel. Scale bars, 200 μm. (FIG. 4B) The OA severity was measured by Mankin score. n=8 mice/group. (FIG. 4C) Average thicknesses of uncalcified (Uncal. Th.) and total (Total Th.) cartilage were quantified at 2 months post-surgery. n=8 mice/group. (FIG. 4D) von Frey assay was performed at 2 months post-surgery. PWT, paw withdrawal threshold. n=8 mice/group. Statistical analysis was performed using one-way ANOVA with Turkey's post hoc analysis for (FIG. 4D) and two-way ANOVA with Turkey's post hoc analysis for (FIG. 4B) and (FIG. 4C). Data presented as means ±SEM. *p<0.05, **p<0.01, ***p<0.001 in (FIG. 4B) and (FIG. 4C). *p<0.05, ***p<0.001 for DMM WT vs. Sham WT;^{$$$}p<0.001 for DMM HBEGF Over^{AgcER} vs. DMM WT;^{&&&}p<0.001 for DMM HBEGF Over^{AgcER} vs. DMM HBEGF Over^{AgcER} Gef in (D).

FIGS. 5A-5I illustrate the preparation and characterization of TGFα-NPs. (FIG. 5A) Schematic diagram of TGFα-NPs. TGFα-NPs were prepared by conjugating TGFα onto polymeric micellar nanoparticles via copper-free click chemistry. (FIG. 5B) DLS measurements of TGFα-NP hydrodynamic diameter (size) and representative image of TGFα-NPs examined by transmission electron microscopy. Scale bar, 100 nm. (FIG. 5C) Zeta potential measurements of TGFα-DBCO, PEG-PCL nanoparticles with or without PLL-PCL, and TGFα-NPs with or without PLL-PCL in 0.1×PBS (PH=7.4). PLL+denotes the nanoparticles that contain PLL-PCL and PLL-denotes the nanoparticles that do not contain PLL-PCL. (FIG. 5D) Stability of TGFα-NPs in water was evaluated by monitoring DLS measurement of TGFα-NP hydrodynamic diameter for up to 7 days. (FIG. 5E) Stability of TGFα-NPs in bovine synovial fluid of knee joint was evaluated by monitoring DLS measurement of TGFα-NP hydrodynamic diameter for up to 24 hours. (FIG. 5F) Cell viability of primary mouse chondrocytes after incubation with TGFα-NPs at different concentrations. (FIG. 5G) Protein levels of EGFR downstream signals (ERK and p-ERK) in articular cartilage chondrocytes induced by different treatments including vehicle, free TGFα, Ctrl-NPs (i.e. no TGFα conjugation), or TGFα-NPs. (FIG. 5H) Quantitative analysis of the relative protein expression level (p-ERK/ERK/β-actin) based on the images of Western blot. n=3 independent experiments. (FIG. 5I) Confocal images of primary chondrocyte treated with vehicle, TGFα-NPs (10 nM TGFα content) or TGFα-NPs (10 nM TGFα content) in the presence of 100 μg/ml free TGFα. Scale bar, 50 μm. Statistical analysis was performed using one-way ANOVA with Dunnett's post hoc analysis. Data presented as means ±SEM. **p<0.01, ***p<0.001.

FIGS. 6A-6F illustrates that TGFα-NPs exhibit full length penetration of human-thickness bovine articular cartilage and extend residence time in both healthy and diseased knee joints. (FIG. 6A) Representative confocal microscopy images of a cross- section of bovine cartilage explants incubated with rhodamine-labeled TGFα-NPs with or without PLL-PCL, or free TGFα for 2, 4 and 6 days. Arrow indicates the diffusion direction. Scale bar, 200 μm. (FIG. 6B) Quantitative analysis of TGFα-NP penetration depth into bovine cartilage explants after 6-day incubation. n=3/group. (FIG. 6C) Quantitative analysis of area under the curve (AUC) based on fluorescence intensity profiles in B. n=3/group. (FIG. 6D) Representative fluorescence images of healthy and OA knee joints over 28 days after intra-articular injection of IRDye 800CW-labeled TGFα or TGFα-NPs. Fluorescent scale: max=3.0×10⁷, min=1.0×10⁸. (FIG. 6E) Quantitative analysis of time course fluorescence radiant efficiency within knee joints after intra-articular injection of IRDye 800CW-labeled TGFα or TGFα-NPs. n=6/group. (FIG. 6F) Quantitative analysis of area under the curve (AUC) based on fluorescence intensity profile in E. n=6/group. Statistical analysis was performed using one-way ANOVA with Turkey's post hoc analysis. Data presented as means ±SEM. ***p<0.001.

FIGS. 7A-7I illustrate that TGFα-NP treatment attenuates OA progression after DMM surgery. (FIG. 7A) Immunostaining of p-EGFR reveals that TGFα-NP treatment enhanced EGFR activity at 1 month post-surgery. Scale bars, 200 μm. (FIG. 7B) Safranin O/Fast Green staining of PBS-, TGFα-DBCO-, Ctrl-NP- or TGFα-NP-treated knee joints at the medial site at 2, and 3 months post-surgery. Low: low magnification image; high: high magnification image of the yellow boxed areas. Scale bars, 200 μm. (FIG. 7C) OA severity of knee joints at 3 months post-surgery was measured by Mankin score. n=8 mice/group. (FIG. 7D) Average uncalcified (Uncal. Th.) cartilage thickness of knee joints at 3 months post-surgery was quantified. n=8 mice/group. (FIG. 7E) Representative 3D color maps show subchondral bone plate thickness (SBP Th.) in the sham and DMM-operated femurs treated with PBS, TGFα-DBCO, Ctrl-NPs and TGFα-NPs. Color ranges from 0 (blue) to 320 μm (red). (FIG. 7F) subchondral bone plate thickness at the medial posterior site of femoral condyle was calculated. n=8 mice/group. (FIG. 7G) H&E staining of mouse knee joints shows changes in synovium at 2 months post-surgery. Red boxed areas indicate the synovium tissues. Scale bar, 200 μm. (FIG. 7H) Synovitis score was measured. n=8 mice/group. (FIG. 7I) von Frey assay was performed at 0, 1, 2, 4, 8, 12 weeks post-surgery. PWT, paw withdrawal threshold. n=8/group. Statistical analysis was performed using one-way ANOVA with Turkey's post hoc analysis. Data presented as means ±SEM. *p<0.05, **p<0.01, ***p<0.001 in (C), (D), (F), (H). *p<0.05, **p<0.01, ***p<0.001 for DMM TGFα-NP vs. DMM PBS in (I).

FIGS. 8A-8B illustrate that HBEGF Over^{Col2} mice have normal body weight and body length. Male HBEGF Over^{Col2} and WT mice at 3 months of age were measured for body weight and length. n=10 mice/group. Statistical analysis was performed using one-way ANOVA with Turkey's post hoc analysis. Data presented as means ±SEM.

FIGS. 9A-9B illustrates that overexpressing HBEGF in articular cartilage does not affect bone structure. (FIG. 9A) Representative longitudinal microCT images of distal femur in WT and HBEGF Over^{Col2} mice at 5 months of age. (FIG. 9B) Trabecular bone structural parameters in the secondary spongiosa were quantified. BMD: bone mineral density, BV/TV: bone volume/tissue volume, Tb.N: trabecular number, Tb.Th: trabecular thickness, Tb. Sp: trabecular separation, SMI: Structure model index. n=5 mice/group. Statistical analysis was performed using one-way ANOVA with Turkey's post hoc analysis. Data presented as means ±SEM.

FIGS. 10A-10B illustrates that overexpressing HBEGF in articular cartilage does not affect cartilage matrix composition and cartilage degradation. (FIG. 10A) Immunostaining of Col II, Col X⁺ and MMP13 in 5-month-old mouse tibial articular cartilage of WT and HBEGF Over^{Col2} mice. Scale bar, 50 μm. (FIG. 10B) Quantification of Col II staining intensity, percentages of Col X⁺ and MMP13⁺ chondrocytes in the tibial articular cartilage of WT and HBEGF Over^{Col2} mice. n=3 mice/group. Statistical analysis was performed using one-way ANOVA with Turkey's post hoc analysis. Data presented as means ±SEM.

FIGS. 11A-11B illustrates that overexpressing HBEGF in cartilage does not affect vital internal organs. (FIG. 11A) Western blot results demonstrate no difference in the protein levels of HBEGF, EGFR, and p-EGFR in the main organs of WT and HBEGF Over^{Col2} mice. Positive control was protein sample from TGFα activated chondrocytes. n=3/group. (FIG. 11B) H&E staining of representative organ sections from WT and HBEGF Over^{Col2} mice. Scale bar, 200 μm. n=5 mice/group.

FIGS. 12A-12B illustrates that HBEGF Over^{AgcER} mice have increased HBEGF expression and EGFR activity in knee articular cartilage. (FIG. 12A) Immunostaining of HBEGF, p-EGFR and EGFR in tibiae of 5-month-old WT and HBEGF Over^{AgcER} mice with Tam injections at 3 months of age. Scale bar, 50 μm. (FIG. 12B) The percentages of HBEGF⁺, p-EGFR⁺ and EGFR⁺ cells within uncalcified articular cartilage were quantified. n=8 mice/group. Statistical analysis was performed using one-way ANOVA with Turkey's post hoc analysis. Data presented as means ±SEM. **p<0.01, ***p<0.001.

FIGS. 13A-13B illustrate the chemical structure (FIG. 13A) and ¹H NMR spectrum (FIG. 13B) of PLL-PCL.

FIG. 14 illustrates that TGFα-NPs result in similar morphology changes in chondrocytes as free TGFα. Primary chondrocytes were treated with vehicle (PBS), free TGFα (15 ng/ml), and TGFα-NP (15 ng/ml TGFα content) for 2 days and imaged by bright field microscopy. Scale bar, 100 μm.

FIGS. 15A-15B illustrate that TGFα-NPs doped with PLL-PCL enhance bovine cartilage uptake. (FIG. 15A) Microscopy images of bovine cartilage explants incubated with free TGFα, or TGFα-NPs with or without PLL-PCL for 24 hours. Fluorescent scale: max =1.5×10⁷, min=2.5×10⁹. (FIG. 15B) Quantitative analysis of TGFα-NPs and free TGFα uptake by bovine cartilage explants based on images in B. n=4/ group. Statistical analysis was performed using one-way ANOVA with Turkey's post hoc analysis. Data presented as means ±SEM. *p<0.05, **p<0.01, ***p<0.001.

FIGS. 16A-16F illustrate that TGFα-NPs doped with PLL-PCL improve their penetration and retention in the bovine cartilage tissue. (FIGS. 16A, 16C, 16E) Quantification of fluorescence intensity of rhodamine labeled TGFα-DBCO (FIG. 16A), TGFα-NPs without PLL-PCL (FIG. 16C) and TGFα-NPs with PLL-PCL (FIG. 16E) across the explant section. All images are taken under the same laser power, intensity and offset. (FIG. 16B, FIG. 16D, FIG. 16F) Area under the curve (AUC) of the corresponding fluorescence intensity from FIG. 16A, FIG. 16C and FIG. 16E. n=3. Statistical analysis was performed using one-way ANOVA with Turkey's post hoc analysis. Data presented as means ±SEM. *p<0.05, **p<0.01, ***p<0.001.

FIGS. 17A-17D illustrate the biodistribution of TGFα-NPs within the knee joints and some major organs. (FIG. 17A) Biodistribution of TGFα-NPs within mouse knee joint. Surrounding tissue include quadriceps, patella, patellar ligament, synovium, fat pad. Fluorescent scale: max=3.0×10⁷, min=1.0×10⁸. (FIG. 17B) Quantification of fluorescent radiant efficiency on different parts of knee joints. n=3 mice/group. (FIG. 17C) Fluorescence images of organs and blood that were collected 24 hours or 1 month after PBS or TGFα-NP injection. Fluorescent scale: max=3.0×10⁷, min=1.0×10⁸. (FIG. 17D) Quantification of fluorescent radiant efficiency on different organs and blood. n=3 mice/group.

FIGS. 18A-18B illustrate the OA severity of knee joints (FIG. 18A) and uncalcified cartilage thickness (FIG. 18B) measured in mice with PBS, TGFα-DBCO, Ctrl-NP and TGFα-NP treatment at 2 months post-surgery. n=8 mice/group. Statistical analysis was performed using one-way ANOVA with Turkey's post hoc analysis. Data presented as means ±SEM. ***p<0.001.

FIGS. 19A-19C illustrate that intra-articular injections of TGFα-NPs into cartilage do not affect vital internal organs and gross joint morphology. (FIG. 19A) H&E staining of representative organ sections from PBS- and TGFα-NP-treated mice. Scale bar, 200 μm. (FIG. 19B) Western blots showed no difference in TGFα, EGFR, and p-EGFR amounts after TGFα-NP injections. Positive control was protein sample from TGFα activated chondrocytes. (FIG. 19C) H&E staining of representative knee joints from PBS-, TGFα-DBCO-, Ctrl-NP- and TGFα-NP-treated mice. Scale bar, 1 mm.

Table 1 illustrates the mouse real-time PCR primer sequences.

Aspects

The following Aspects are illustrative only and do not limit the scope of the present disclosure or the appended claims.

Aspect 1. A therapeutic composition, comprising: a polymeric nanoparticle; a ligand selected to activate an EGFR receptor; and a linker, the linker associating the nanoparticle and the ligand.

Aspect 2. The therapeutic composition of Aspect 1, wherein the ligand is one or more of EGF, transforming growth factor-alpha (TGFα), heparin-binding EGF-like growth factor (HBEGF), betacellulin (BTC), amphiregulin (AREG), epiregulin (EREG), or epigen.

Aspect 3. The therapeutic composition of Aspect 2, wherein the ligand is TGFα.

Aspect 4. The therapeutic composition of Aspect 1, wherein the ligand differs from a naturally-occurring ligand by one or more amino acids. Such a difference can be a synthetic one, e.g., via substituting an amino acid for an amino acid that occurs in the naturally-occurring ligand, via adding an additional amino acid to the naturally-occurring ligand, via removing an amino acid from the naturally-occurring ligand, or any combination thereof. Example synthetic ligands include, e.g., epidermal growth factor (EGF), and heparin-binding EGF-like growth factor (HBEGF). A bioconjugatable group can be comprised with a ligand; example bioconjugatable groups include (without limitation) amine, carboxyl, and thiol. As described elsewhere herein, a bioconjugatable group can be used to conjugate the ligand to a nanoparticle.

Aspect 5. The therapeutic composition of any one of Aspects 1-4, wherein the polymeric nanoparticle comprises at least (1) a first polymer; (2) a second polymer, the second polymer comprising at least one positively charged group; and (3) an anchor species that associates with the linker.

Aspect 6. The therapeutic composition of Aspect 5, wherein the first polymer comprises PEG, PCL, dextran, poly (D,L-lactic acid) (PLA), poly (D,L-lactic-co-glycolic acid) (PLGA), a phospholipid, or any combination thereof.

Aspect 7. The therapeutic composition of Aspect 6, wherein the first polymer comprises a PEG-PCL diblock copolymer, the PEG-PCL diblock copolymer optionally having a molecular weight in the range of from about 3000 to about 30,000, e.g., from about 3000 to about 30,000, from about 5000 to about 25,000, from about 7500 to about 20,000, from about 10,000 to about 15,000, and all intermediate values.

Aspect 8. The therapeutic composition of any one of Aspects 5-7, wherein the second polymer comprises either or both of PLL and N-[1-(2,3-Dioleoyloxy)propyl]-N,N,N-trimethylammonium methyl-sulfate (DOTAP).

Aspect 9. The therapeutic composition of Aspect 8, wherein the second polymer comprises a PLL-PCL diblock copolymer, the PLL-PCL diblock copolymer optionally having a molecular weight in the range of from about 1500 to about 30,000, e.g., from about 1500 to about 30,000, from about 2000 to about 25,000, from about 3000 to about 20,000, from about 5000 to about 15,000, or even about 10,000.

Aspect 10. The therapeutic composition of any one of Aspects 1-9, wherein the polymeric nanoparticle is characterized as having a surface charge of from about −5 mV to about 30 mV. The surface charge can be, e.g., from −5 to 30 mV, from −4.5 to 28 mV, from −4.3 to 26 mV, from −4.1 to 24 mV, from −3.8 to 22 mV, from −3.5 to 20 mV, from −3.2 to 18 mV, from −3 to 16 mV, from −2.8 to 14 mV, from −2.6 to 12 mV, from −2.3 to 10 mV, from −2.1 to 8 mV, from −1.9 to 7 mV, from −1.7 to 6 mV, from −1.5 to 5 mV, from −1.3 to 4 mV, or even from −0.9 to 3 mV.

Aspect 11. The therapeutic composition of any one of Aspects 1-10, wherein the therapeutic composition is characterized as having a surface charge of from about −5 to about 30 mV.

Aspect 12. The therapeutic composition of any one of Aspects 1-11, wherein the linker covalently associates the ligand and the nanoparticle via click chemistry.

Aspect 13. The therapeutic composition of any one of Aspects 1-12, wherein the therapeutic composition is characterized as having a hydrodynamic diameter in the range of from about 10 to about 80 nm. Example diameters are, e.g., from about 10 to about 80 nm, from about 15 to about 75 nm, from about 20 to about 70 nm, from about 25 to about 65 nm, from about 30 to about 60 nm, from about 35 to about 55 nm, or even from about 40 to about 50 nm, and all intermediate values and sub-ranges.

Aspect 14. The therapeutic composition of Aspect 13, wherein the hydrodynamic diameter remains essentially unchanged following the therapeutic composition's exposure to water for 1 week.

Aspect 15. A therapeutic composition, comprising: a nanoparticle; a ligand, the ligand being any one of EGF, transforming growth factor-alpha (TGFα), heparin-binding EGF-like growth factor (HBEGF), betacellulin (BTC), amphiregulin (AREG), epiregulin (EREG), or epigen; and a linker associating the nanoparticle and the ligand, the therapeutic composition having a surface charge in the range of from about −5 to about 30 mV.

The surface charge can be, e.g., from −5 to 30 mV, from −4.5 to 28 mV, from −4.3 to 26 mV, from −4.1 to 24 mV, from −3.8 to 22 mV, from −3.5 to 20 mV, from −3.2 to 18 mV, from −3 to 16 mV, from −2.8 to 14 mV, from −2.6 to 12 mV, from −2.3 to 10 mV, from −2.1 to 8 mV, from −1.9 to 7 mV, from −1.7 to 6 mV, from −1.5 to 5 mV, from −1.3 to 4 mV, or even from −0.9 to 3 mV.

Aspect 16. The therapeutic composition of Aspect 15, wherein the ligand is TGFα.

Aspect 17. The therapeutic composition of any one of Aspects 15-16, wherein the nanoparticle comprises a polymer, a phospholipid, a dendrimer, glycol chitosan, or any combination thereof.

Aspect 18. The therapeutic composition of any one of Aspects 15-16, wherein the nanoparticle comprises at least (1) a first polymer; (2) a second polymer, the second polymer comprising at least one charged group; and (3) an anchor species that associates with the linker.

Aspect 19. A method of treating joint pain in a patient in need thereof, the method comprising: administering to the patient a therapeutically effective amount of a composition comprising the therapeutic composition of any one of Aspects 1-14 or any one of Aspects 15-18.

Aspect 20. The method of Aspect 19, wherein the administering is performed following a surgery to the joint.

Aspect 21. The method of Aspect 19, wherein the administering is performed to a nonsurgical patient.

Aspect 22. The method of any one of Aspects 19-21, wherein the joint is a foot joint, an ankle joint, a knee joint, a hip joint, a hand joint, an elbow joint, or a shoulder joint.

Aspect 23. A pharmaceutically acceptable composition, comprising the therapeutic composition of any one of Aspects 1-14 or any one of Aspects 15-18 and a pharmaceutically acceptable excipient.

### References

R. F. Loeser, S. R. Goldring, C. R. Scanzello, M. B. Goldring, Osteoarthritis: a disease of the joint as an organ. Arthritis Rheum. 64, 1697-1707. (2012).

J. Becerra, J. A. Andrades, E. Guerado, P. Zamora-Navas, J. M. Lopez-Puertas, A. H. Reddi, Articular cartilage: structure and regeneration. Tissue Eng Part B Rev. 16, 617-627. (2010).

S. S. Glasson, M. G. Chambers, W. B. Van Den Berg, C. B. Little, The OARSI histopathology initiative—recommendations for histological assessments of osteoarthritis in the mouse. Osteoarthritis Cartilage. 18, S17-23. (2010).

K. P. Pritzker, S. Gay, S. A. Jimenez, K. Ostergaard, J. P. Pelletier, P. A. Revell, D. Salter, W. B. van den Berg, Osteoarthritis cartilage histopathology: grading and staging. Osteoarthritis Cartilage. 14, 13-29. (2006).

B. Rolauffs, M. Rothdiener, C. Bahrs, A. Badke, K. Weise, K. E. Kuettner, B. Kurz, M. Aurich, A. J. Grodzinsky, W. K. Aicher, Onset of preclinical osteoarthritis: the angular spatial organization permits early diagnosis. Arthritis Rheum. 63, 1637-1647. (2011).

A. Citri, Y. Yarden, EGF-ERBB signalling: towards the systems level. Nat Rev Mol Cell Biol. 7, 505-516. (2006).

H. Jia, X. Ma, W. Tong, B. Doyran, Z. Sun, L. Wang, X. Zhang, Y. Zhou, F. Badar, A. Chandra, X. L. Lu, Y. Xia, L. Han, M. Enomoto-Iwamoto, L. Qin, EGFR signaling is critical for maintaining the superficial layer of articular cartilage and preventing osteoarthritis initiation. Proc Natl Acad Sci USA. 113, 14360-14365. (2016).

H. Jia, X. Ma, Y. Wei, W. Tong, R. J. Tower, A. Chandra, L. Wang, Z. Sun, Z. Yang, F. Badar, K. Zhang, W. J. Tseng, I. Kramer, M. Kneissel, Y. Xia, X. S. Liu, J. H. Wang, L. Han, M. Enomoto-Iwamoto, L. Qin, Loading-Induced Reduction in Sclerostin as a Mechanism of Subchondral Bone Plate Sclerosis in Mouse Knee Joints During Late-Stage Osteoarthritis. Arthritis Rheumatol. 70, 230-241. (2018).

X. Zhang, K. A. Pickin, R. Bose, N. Jura, P. A. Cole, J. Kuriyan, Inhibition of the EGF receptor by binding of MIG6 to an activating kinase domain interface. Nature. 450, 741-744. (2007).

M. A. Pest, B. A. Russell, Y. W. Zhang, J. W. Jeong, F. Beier, Disturbed cartilage and joint homeostasis resulting from a loss of mitogen-inducible gene 6 in a mouse model of joint dysfunction. Arthritis Rheumatol. 66, 2816-2827. (2014).

J. B. Shepard, J. W. Jeong, N. J. Maihle, S. O'Brien, C. N. Dealy, Transient anabolic effects accompany epidermal growth factor receptor signal activation in articular cartilage in vivo. Arthritis Res Ther. 15, R60. (2013).

B. Staal, B. O. Williams, F. Beier, G. F. Vande Woude, Y. W. Zhang, Cartilage-specific deletion of Mig-6 results in osteoarthritis-like disorder with excessive articular chondrocyte proliferation. Proc Natl Acad Sci USA 111, 2590-2595 (2014).

T. Buch, F. L. Heppner, C. Tertilt, T. J. Heinen, M. Kremer, F. T. Wunderlich, S. Jung, A. Waisman, A Cre-inducible diphtheria toxin receptor mediates cell lineage ablation after toxin administration. Nat Methods. 2, 419-426. (2005).

R. Iwamoto, S. Higashiyama, T. Mitamura, N. Taniguchi, M. Klagsbrun, E. Mekada, Heparin-binding EGF-like growth factor, which acts as the diphtheria toxin receptor, forms a complex with membrane protein DRAP27/CD9, which up-regulates functional receptors and diphtheria toxin sensitivity. EMBO J. 13, 2322-2330. (1994).

W. Pan, K. Vallance, A. J. Kastin, TGFalpha and the blood-brain barrier: accumulation in cerebral vasculature. Exp Neurol. 160, 454-459. (1999).

B. C. Geiger, S. Wang, R. F. Padera, Jr., A. J. Grodzinsky, P. T. Hammond, Cartilage-penetrating nanocarriers improve delivery and efficacy of growth factor treatment of osteoarthritis. Sci Transl Med 10, eaat8800 (2018).

S. Kumar, I. M. Adjei, S. B. Brown, 0. Liseth, B. Sharma, Manganese dioxide nanoparticles protect cartilage from inflammation-induced oxidative stress. Biomaterials 224, 119467 (2019).

H. Yan, X. Duan, H. Pan, N. Holguin, M. F. Rai, A. Akk, L. E. Springer, S. A. Wickline, L. J. Sandell, C. T. Pham, Suppression of NF-kappaB activity via nanoparticle-based siRNA delivery alters early cartilage responses to injury. Proc Natl Acad Sci USA 113, E6199-E6208 (2016).

E. Kozhemyakina, M. Zhang, A. Ionescu, U. M. Ayturk, N. Ono, A. Kobayashi, H. Kronenberg, M. L. Warman, A. B. Lassar, Identification of a Prg4-expressing articular cartilage progenitor cell population in mice. Arthritis Rheumatol. 67, 1261-1273. (2015).

U. Sahin, G. Weskamp, K. Kelly, H. M. Zhou, S. Higashiyama, J. Peschon, D. Hartmann, P. Saftig, C. P. Blobel, Distinct roles for ADAM10 and ADAM17 in ectodomain shedding of six EGFR ligands. J Cell Biol. 164, 769-779 (2004).

B. Doyran, W. Tong, Q. Li, H. Jia, X. Zhang, C. Chen, M. Enomoto-Iwamoto, X. L. Lu, L. Qin, L. Han, Nanoindentation modulus of murine cartilage: a sensitive indicator of the initiation and progression of post-traumatic osteoarthritis. Osteoarthritis Cartilage. 25, 108-117. (2017).

K. Elenius, S. Paul, G. Allison, J. Sun, M. Klagsbrun, Activation of HER4 by heparin-binding EGF-like growth factor stimulates chemotaxis but not proliferation. EMBO J. 16, 1268-1278. (1997).

C. Yewale, D. Baradia, I. Vhora, S. Patil, A. Misra, Epidermal growth factor receptor targeting in cancer: a review of trends and strategies. Biomaterials. 34, 8690-8707 (2013).

X. Zhang, J. Zhu, F. Liu, Y. Li, A. Chandra, L. S. Levin, F. Beier, M. Enomoto-Iwamoto, L. Qin, Reduced EGFR signaling enhances cartilage destruction in a mouse osteoarthritis model. Bone Research 2, 14015 (2014).

B. Singh, R. J. Coffey, From wavy hair to naked proteins: the role of transforming growth factor alpha in health and disease. Semin Cell Dev Biol. 28, 12-21 (2014).

J. Z. Hui, A. Al Zaki, Z. Cheng, V. Popik, H. Zhang, E. T. Luning Prak, A. Tsourkas, Facile method for the site-specific, covalent attachment of full-length IgG onto nanoparticles. Small 10, 3354-3363 (2014).

R. Warden-Rothman, I. Caturegli, V. Popik, A. Tsourkas, Sortase-tag expressed protein ligation: combining protein purification and site-specific bioconjugation into a single step. Anal. Chem 85, 11090-11097 (2013).

A. J. Sophia Fox, A. Bedi, S. A. Rodeo, The basic science of articular cartilage: structure, composition, and function. Sports Health. 1, 461-468 (2009).

A. Mathiessen, P. G. Conaghan, Synovitis in osteoarthritis: current understanding with therapeutic implications. Arthritis Res Ther. 19, 18. (2017).

L. Qin, F. Beier, EGFR Signaling: Friend or Foe for Cartilage? JBMR Plu 3, e10177 (2019).

T. Uchimura, A. T. Foote, E. L. Smith, E. G. Matzkin, L. Zeng, Insulin-Like Growth Factor II (IGF-II) Inhibits IL-lbeta-Induced Cartilage Matrix Loss and Promotes Cartilage Integrity in Experimental Osteoarthritis. J Cell Biochem. 116, 2858-2869. (2015).

E. E. Moore, A. M. Bendele, D. L. Thompson, A. Littau, K. S. Waggie, B. Reardon, J. L. Ellsworth, Fibroblast growth factor-18 stimulates chondrogenesis and cartilage repair in a rat model of injury-induced osteoarthritis. Osteoarthritis Cartilage. 13, 623-631. (2005).

Y. I. Jeong, J. B. Cheon, S. H. Kim, J. W. Nah, Y. M. Lee, Y. K. Sung, T. Akaike, C. S. Cho, Clonazepam release from core-shell type nanoparticles in vitro. J Control Release 51, 169-178 (1998).

A. Al Zaki, D. Joh, Z. L. Cheng, A. L. B. De Barros, G. Kao, J. Dorsey, A. Tsourkas, Gold-Loaded Polymeric Micelles for Computed Tomography-Guided Radiation Therapy Treatment and Radiosensitization. Acs Nano 8, 104-112 (2014).

A. V. Kabanov, E. V. Batrakova, N. S. Meliknubarov, N. A. Fedoseev, T. Y. Dorodnich, V. Y. Alakhov, V. P. Chekhonin, I. R. Nazarova, V. A. Kabanov, A New Class of Drug Carriers - Micelles of Poly(Oxyethylene)-Poly(Oxypropylene) Block Copolymers as Microcontainers for Drug Targeting from Blood in Brain. J Control Release 22, 141-157 (1992).

H. Cabral, K. Miyata, K. Osada, K. Kataoka, Block Copolymer Micelles in Nanomedicine Applications. Chem Rev. 118, 6844-6892. (2018).

C. T. Appleton, S. E. Usmani, S. M. Bernier, T. Aigner, F. Beier, Transforming growth factor alpha suppression of articular chondrocyte phenotype and Sox9 expression in a rat model of osteoarthritis. Arthritis Rheum. 56, 3693-3705. (2007).

X. Zhang, J. Zhu, Y. Li, T. Lin, V. A. Siclari, A. Chandra, E. M. Candela, E. Koyama, M. Enomoto-Iwamoto, L. Qin, Epidermal growth factor receptor (EGFR) signaling regulates epiphyseal cartilage development through beta-catenin-dependent and -independent pathways. J Biol Chem 288, 32229-32240 (2013).

D. L. Long, V. Ulici, S. Chubinskaya, R. F. Loeser, Heparin-binding epidermal growth factor-like growth factor (HB-EGF) is increased in osteoarthritis and regulates chondrocyte catabolic and anabolic activities. Osteoarthritis Cartilage. 23, 1523-1531. (2015).

Y. W. Zhang, Y. Su, N. Lanning, P. J. Swiatek, R. T. Bronson, R. Sigler, R. W. Martin, G. F. Vande Woude, Targeted disruption of Mig-6 in the mouse genome leads to early onset degenerative joint disease. Proc Natl Acad Sci USA 102, 11740-11745 (2005).

P. Li, Q. Deng, J. Liu, J. Yan, Z. Wei, Z. Zhang, H. Liu, B. Li, Roles for HB-EGF in Mesenchymal Stromal Cell Proliferation and Differentiation During Skeletal Growth. J Bone Miner Res. 34, 295-309. (2019).

K. Yu, J. Xu, Z. Liu, D. Sosic, J. Shao, E. N. Olson, D. A. Towler, D. M. Ornitz, Conditional inactivation of FGF receptor 2 reveals an essential role for FGF signaling in the regulation of osteoblast function and bone growth. Development. 130, 3063-3074 (2003).

D. A. Ovchinnikov, J. M. Deng, G. Ogunrinu, R. R. Behringer, Col2a1-directed expression of Cre recombinase in differentiating chondrocytes in transgenic mice. Genesis. 26, 145-146. (2000).

S. P. Henry, C. W. Jang, J. M. Deng, Z. Zhang, R. R. Behringer, B. de Crombrugghe, Generation of aggrecan-CreERT2 knockin mice for inducible Cre activity in adult cartilage. Genesis. 47, 805-814. (2009).

G. Pante, J. Thompson, F. Lamballe, T. Iwata, I. Ferby, F. A. Barr, A. M. Davies, F. Maina, R. Klein, Mitogen-inducible gene 6 is an endogenous inhibitor of HGF/Met-induced cell migration and neurite growth. J Cell Biol. 171, 337-348. (2005).

M. K. Lotz, S. Otsuki, S. P. Grogan, R. Sah, R. Terkeltaub, D. D'Lima, Cartilage cell clusters. Arthritis Rheum. 62, 2206-2218. (2010).

R. Roskoski, Jr., The ErbB/HER family of protein-tyrosine kinases and cancer. Pharmacol Res. 79, 34-74 (2014).

J. Z. Hui, A. Al Zaki, Z. Cheng, V. Popik, H. Zhang, E. T. Luning Prak, A. Tsourkas, Facile method for the site-specific, covalent attachment of full-length IgG onto nanoparticles. Small. 10, 3354-3363. (2014).

X. Zhang, J. Zhu, Y. Li, T. Lin, V. A. Siclari, A. Chandra, E. M. Candela, E. Koyama, M. Enomoto-Iwamoto, L. Qin, Epidermal growth factor receptor (EGFR) signaling regulates epiphyseal cartilage development through beta-catenin-dependent and -independent pathways. J Biol Chem. 288, 32229-32240 (2013).

V. Krenn, L. Morawietz, T. Haupl, J. Neidel, I. Petersen, A. Konig, Grading of chronic synovitis--a histopathological grading system for molecular and diagnostic pathology. Pathol Res Pract 198, 317-325. (2002).

T. Aigner, J. L. Cook, N. Gerwin, S. S. Glasson, S. Laverty, C. B. Little, W. McIlwraith, V. B. Kraus, Histopathology atlas of animal model systems - overview of guiding principles. Osteoarthritis Cartilage 18 Suppl 3, S2-6 (2010).

M. A. Batista, H. T. Nia, P. Onnerfjord, K. A. Cox, C. Ortiz, A. J. Grodzinsky, D. Heinegard, L. Han, Nanomechanical phenotype of chondroadherin-null murine articular cartilage. Matrix Biol. 38, 84-90. (2014).

M. J. Piel, J. S. Kroin, H. J. Im, Assessment of knee joint pain in experimental rodent models of osteoarthritis. Methods Mol Biol. 1226, 175-181. (2015).

A. Chandra, S. Lan, J. Zhu, V. Siclari, L. Qin, Epidermal Growth Factor Receptor (EGFR) signaling promotes proliferation and survival in osteoprogenitors by increasing Early Growth Response Protein (Egr2) expression. J Biol Chem. 288, 20488-98. (2013).

1. A therapeutic composition, comprising:
a polymeric nanoparticle;
a ligand selected to activate an EGFR receptor; and
a linker,
the linker associating the nanoparticle and the ligand.

2. The therapeutic composition of claim 1, wherein the ligand is EGF, transforming growth factor-alpha (TGFα), heparin-binding EGF-like growth factor (HBEGF), betacellulin (BTC), amphiregulin (AREG), epiregulin (EREG), or epigen.

3. The therapeutic composition of claim 2, wherein the ligand is TGFα.

4. The therapeutic composition of claim 1, wherein the ligand differs from a naturally-occurring ligand by one or more amino acids.

5. The therapeutic composition of claim 1, wherein the polymeric nanoparticle comprises at least (1) a first polymer; (2) a second polymer, the second polymer comprising at least one positively charged group; and (3) an anchor species that associates with the linker.

6. The therapeutic composition of claim 5, wherein the first polymer comprises PEG, PCL, dextran, poly (D,L-lactic acid) (PLA), poly (D,L-lactic-co-glycolic acid) (PLGA), a phospholipid, or any combination thereof.

7. The therapeutic composition of claim 6, wherein the first polymer comprises a PEG-PCL diblock copolymer, the PEG-PCL diblock copolymer optionally having a molecular weight in the range of from about 3000 to about 30,000.

8. The therapeutic composition of claim 5, wherein the second polymer comprises PLL or N-[1-(2,3-Dioleoyloxy)propyl]-N,N,N-trimethylammonium methyl-sulfate (DOTAP).

9. The therapeutic composition of claim 8, wherein the second polymer comprises a PLL-PCL diblock copolymer, the PLL-PCL diblock copolymer optionally having a molecular weight in the range of from about 1500 to about 30,000.

10. The therapeutic composition of claim 1, wherein the polymeric nanoparticle is characterized as having a surface charge of from about -5 mV to about 30 mV.

11. The therapeutic composition of claim 1, wherein the therapeutic composition is characterized as having a surface charge of from about −5 to about 30 mV.

12. The therapeutic composition of claim 1, wherein the linker covalently associates the ligand and the nanoparticle via click chemistry.

13. The therapeutic composition of claim 1, wherein the therapeutic composition is characterized as having a hydrodynamic diameter in the range of from about 10 to about 80 nm.

14. The therapeutic composition of claim 13, wherein the hydrodynamic diameter remains essentially unchanged following the therapeutic composition's exposure to water for 1 week.

15. A therapeutic composition, comprising:
a nanoparticle;
a ligand, the ligand being any one of EGF, transforming growth factor-alpha (TGFα), heparin-binding EGF-like growth factor (HBEGF), betacellulin (BTC), amphiregulin (AREG), epiregulin (EREG), or epigen; and
a linker associating the nanoparticle and the ligand,
the therapeutic composition having a surface charge in the range of from about −5 to about 30 mV.

16. The therapeutic composition of claim 15, wherein the ligand is TGFα.

17. The therapeutic composition of claim 15, wherein the nanoparticle comprises a polymer, a phospholipid, a dendrimer, glycol chitosan, or any combination thereof.

18. The therapeutic composition of claim 15, wherein the nanoparticle comprises at least (1) a first polymer; (2) a second polymer, the second polymer comprising at least one charged group; and (3) an anchor species that associates with the linker.

19. A method of treating joint pain in a patient in need thereof, the method comprising: administering to the patient a therapeutically effective amount of a composition comprising the therapeutic composition of claim 1.

20. The method of claim 19, wherein the administering is performed following a surgery to the joint.

21. The method of claim 19, wherein the administering is performed to a nonsurgical patient.

22. The method of claim 19, wherein the joint is a foot joint, an ankle joint, a knee joint, a hip joint, a hand joint, an elbow joint, or a shoulder joint.

23. A pharmaceutically acceptable composition, comprising the therapeutic composition of claim 1 and a pharmaceutically acceptable excipient.

24. A method of treating joint pain in a patient in need thereof, the method comprising: administering to the patient a therapeutically effective amount of a composition comprising the therapeutic composition of claim 15.

25. The method of claim 24, wherein the administering is performed following a surgery to the joint.

26. The method of claim 24, wherein the administering is performed to a nonsurgical patient.

27. The method of claim 24, wherein the joint is a foot joint, an ankle joint, a knee joint, a hip joint, a hand joint, an elbow joint, or a shoulder joint.

28. A pharmaceutically acceptable composition, comprising the therapeutic composition of claim 15 and a pharmaceutically acceptable excipient.


SEQUENCE LISTING (19 sequences)

SEQ ID NO: 1 - protein, 12 aa, Artificial Sequence
  Description of Artificial Sequence Synthetic 12xHis tag
HHHHHHHHHHHH

SEQ ID NO: 2 - DNA, 20 bp, Artificial Sequence
  Description of Artificial Sequence Synthetic primer
ctatgaccacacaaccatcc

SEQ ID NO: 3 - DNA, 22 bp, Artificial Sequence
  Description of Artificial Sequence Synthetic primer
ggaaaccgaaatttgtgctacg

SEQ ID NO: 4 - DNA, 20 bp, Artificial Sequence
  Description of Artificial Sequence Synthetic primer
caggagccaatgaagaagtg

SEQ ID NO: 5 - DNA, 21 bp, Artificial Sequence
  Description of Artificial Sequence Synthetic primer
caagaacagcaacgagtaccg

SEQ ID NO: 6 - DNA, 19 bp, Artificial Sequence
  Description of Artificial Sequence Synthetic primer
ctaccgctgtgaagtgatg

SEQ ID NO: 7 - DNA, 21 bp, Artificial Sequence
  Description of Artificial Sequence Synthetic primer
gggagtttggctccagagttt

SEQ ID NO: 8 - DNA, 20 bp, Artificial Sequence
  Description of Artificial Sequence Synthetic primer
tgacctccacagttgacagg

SEQ ID NO: 9 - DNA, 22 bp, Artificial Sequence
  Description of Artificial Sequence Synthetic primer
tcctcctgagcgcaagtactct

SEQ ID NO: 10 - DNA, 22 bp, Artificial Sequence
  Description of Artificial Sequence Synthetic primer
tcctctcctatggtacctaaac

SEQ ID NO: 11 - DNA, 22 bp, Artificial Sequence
  Description of Artificial Sequence Synthetic primer
gccttgcagtctttctcagctc

SEQ ID NO: 12 - DNA, 20 bp, Artificial Sequence
  Description of Artificial Sequence Synthetic primer
gaaggtgtgtgtctggaaag

SEQ ID NO: 13 - DNA, 21 bp, Artificial Sequence
  Description of Artificial Sequence Synthetic primer
gtcactggtcaactccagcac

SEQ ID NO: 14 - DNA, 20 bp, Artificial Sequence
  Description of Artificial Sequence Synthetic primer
ggtgtagcgtgtggaaatag

SEQ ID NO: 15 - DNA, 21 bp, Artificial Sequence
  Description of Artificial Sequence Synthetic primer
tgtgtcttcaggggtccttag

SEQ ID NO: 16 - DNA, 21 bp, Artificial Sequence
  Description of Artificial Sequence Synthetic primer
atcaggcactccacatcttgg

SEQ ID NO: 17 - DNA, 22 bp, Artificial Sequence
  Description of Artificial Sequence Synthetic primer
cggactcatcgtactcctgctt

SEQ ID NO: 18 - protein, 5 aa, Artificial Sequence
  Description of Artificial Sequence Synthetic peptide
  MOD_RES (3)..(3): Any amino acid
LPXTG

SEQ ID NO: 19 - protein, 5 aa, Artificial Sequence
  Description of Artificial Sequence Synthetic peptide
  MOD_RES (5)..(5): Lys-DBCO
GGGSK